(19) United States
(12) Patent Application Publication
(10) Pub. No.: US 2016/0331521 A1
(43) Pub. Date: Nov. 17, 2016

(54) ACCOMMODATION-RESPONSIVE INTRAOCULAR LENSES

(71) Applicants: Charles DEBOER, Sierra Madre, CA (US); Craig Alan CABLE II, Mission Viejo, CA (US); Mark S HUMAYUN, Glendale, CA (US); Yu-Chong TAI, Pasadena, DC (US); Sean CAFFEY, Pasadena, CA (US); Wendian SHI, Monrovia, CA (US); Andrew URAZAKI, Arcadia, CA (US)

(72) Inventors: Charles DEBOER, Sierra Madre, CA (US); Craig Alan CABLE II, Mission Viejo, CA (US); Mark S HUMAYUN, Glendale, CA (US); Yu-Chong TAI, Pasadena, DC (US); Sean CAFFEY, Pasadena, CA (US); Wendian SHI, Monrovia, CA (US); Andrew URAZAKI, Arcadia, CA (US)

(21) Appl. No.: 15/139,129

(22) Filed: Apr. 26, 2016

Related U.S. Application Data
(60) Provisional application No. 62/159,620, filed on May 11, 2015.
(60) Provisional application No. 62/159,661, filed on May 11, 2015.
(60) Provisional application No. 62/161,302, filed on May 14, 2015.

(51) Int. Cl. A61F 2/16

(52) U.S. Cl. CPC A61F 2/1635; A61F 2002/1683

(57) ABSTRACT

Fluid-filled accommodating intraocular lenses include components enabling the lens to effectively respond to the eye's natural accommodation process, thereby allowing the patient to visualize over a range of focal distances with minimal complications. Internal components may include, for example, a rigid member that alters optical power of the lens and/or a spanning member extending across the lens that affects the response to accommodative action and/or to filling, or overfilling, of the lens with an optical fluid. Various combinations of internal and external components may be implanted in distinct successive steps or during separate operations to minimize complications and incision size.

### CROSS-REFERENCE TO RELATED APPLICATIONS

This application claims priority to, and the benefits of. U.S. Provisional Patent Application No. 62/159,620, filed on May 11, 2015, U.S. Provisional Patent Application No. 62/159,638, filed on May 11, 2015, U.S. Provisional Patent Application No. 62/159,661, filed on May 11, 2015, and U.S. Provisional Patent Application No. 62/161,302, filed on May 14, 2015, the entire disclosures of which are hereby incorporated by reference.

### FIELD OF THE INVENTION

The present invention relates generally to implantable intraocular lenses for vision correction.

### BACKGROUND

The crystalline lens of the human eye refracts and focuses light onto the retina. Normally the lens is clear, but it can become opaque (i.e., when developing a cataract) due to aging, trauma, inflammation, metabolic or nutritional disorders, or radiation. While some lens opacities are small and require no treatment, others may be large enough to block significant fractions of light and obstruct vision.

Conventionally, cataract treatments involve surgically removing the opaque lens matrix from the lens capsule using, for example, phacoemulsification and/or a femtosecond laser through a small incision in the periphery of the patient's cornea. An artificial intraocular lens (IOL) can then be implanted in the lens capsular bag (or “capsule bag”)—the sack-like structure remaining within the eye following extracapsular cataract extraction; the lens “capsule” is the thin clear membrane that surrounds the natural crystalline lens—to replace the natural lens. Generally, IOLs are made of a foldable material, such as silicone or uncrosslinked acrylics, to minimize the incision size and required stitches and, as a result, the patient's recovery time. The most commonly used IOLs are single-element lenses (or monofocal IOLs) that provide a single focal distance; the selected focal length typically affords fairly good distance vision. However, because the focal distance is not adjustable following implantation of the IOL, patients implanted with monofocal IOLs can no longer focus on objects at to close distance (e.g., less than 60 cm); this results in poor visual acuity at close distances. To negate this disadvantage, multifocal IOLs provide dual foci at both near and far distances. However, due to the optical design of such lenses, patients implanted with multifocal IOLs often suffer from a loss of vision sharpness (e.g., blurred vision, halos, glare, and decreased contrast sensitivity). In addition, patients may experience visual disturbances, such as halos or glare, because of the simultaneous focus at two distances.

Recently, accommodating intraocular lenses (AIOLs) have been developed to provide adjustable focal distances (or “accommodations”), relying on the natural focusing ability of the eye. The term “accommodation” generally refers to the process by which the eye changes optical power to maintain focus at different distances, e.g., as an object recedes or approaches. When the circular ciliary muscle relaxes, the fibrous zonules that connect the muscle to the lens pull on the lens, flattening it to focus on a far object. When accommodating to a near object, the ciliary muscles contract and the lens zonules slacken, allowing the lens to assume a thicker and more convex form.

AIOLs respond to this ocular behavior in as manner analogous to that of the natural lens. Conventional AIOLs include, for example, a single optic that translates its position along the visual axis of the eye, dual optics that change the distance between two lenses, and curvature-changing lenses that change their curvatures to adjust the focus power. These designs, however, tend to be too complex to be practical to construct and/or have achieved limited success (e.g., providing a focusing power of only 1-2 diopters). One reason for the lack of success is the fact that the AIOL may not respond mechanically the way the natural lens does, or the patient may have an ocular anatomy that requires a non-uniform response by the lens.

Consequently, there is still a need for AIOLs that provide a high degree of accommodation and provide appropriate focusing power, optically respond correctly to the natural focusing mechanism of the patient's eye, and which can be easily manufactured and implanted in human eyes. In addition, such AIOLs should respond appropriately to the eye's natural accommodation mechanism, thereby allowing the patient to experience a range of focal distances with minimal complications.

### SUMMARY

In various embodiments, the invention relates to an AIOL that corrects vision and is optically responsive to the natural focusing mechanism of the patient's eye. The AIOL may include one or more components internal to its “lens” (typically a fluid-fillable reservoir having an exterior flexible membrane) as well as one or more components external to the lens.

In certain embodiments, an internal component includes, consists essentially of, or consists of a spanning member extending across the lens and that affects the way the device responds to accommodative action and/or to filling, or overfilling, of the lens with an optical fluid. Although internal component(s) may be located in the optical portion of the lens—i.e., a central chamber portion that effects vision correction following implantation—it or they may be configured to avoid interfering with the patient's vision. A spanning member may act to restrain lens expansion, in the manner of a rope, or may resist lens contraction in the manner of a strut.

In various other embodiments, an internal component includes, consists essentially of, or consists of an optically shaped rigid component. The rigid component comes into contact with the fluid-filled lens as the eye focuses, causing the fluid-filled lens to change shape. (As used herein, the terms “fluid-filled” and “fluid-fillable” are interchangeable and refer to a lens comprising or consisting essentially of an optically transparent and typically flexible membrane that defines a reservoir fillable by fluid; the fluid and membrane at least partially dictate the optical power of the lens. The lens is not necessarily filled with fluid prior to implantation in a patient's eye.) Physically the rigid component may be mounted to the lens through a series of flexible coupling members, which allow it to move in the anterior-posterior direction.

The rigid member may have optical focusing ability such as a lens, or it may have no optical power, as in the case that it is a uniform thickness optically clear spherical (or other shape—e.g. asphetical, toric, multifocal, planar) shell. In all cases, the rigid member is free to disengage from the lens in the axial direction, or to engage and alter the anterior (or posterior) surface of the lens. This movement is actuated either by directly applying pressure to the rigid member or by applying a force to the flexible members.

In various embodiments, the rigid component is located, either anterior to the anterior surface of the lens (or posterior to the posterior lens surface). The rigid component typically has a surface curvature that differs from curvature of the fluid-filled lens. As it comes into contact with the anterior surface of the fluid-filled lens, it causes the fluid-filled lens to conform to its curvature. Therefore, actuation of the rigid component causes an optical power change in the fluid-filled lens itself due to a curvature change of the anterior surface of the fluid-filled lens.

In some embodiments, the rigid member is a spherical-shaped portion with a radius of curvature larger than the liquid filled intraocular lens. When the fluid-filled lens contacts the rigid member, it assumes its shape and overall fluid-filled lens power is decreased due to a decrease in optical power from the anterior surface of the lens. When the rigid member is not in contact with the lens, the anterior surface of the fluid-filled lens takes its nominal shape and optical power is higher, corresponding to the accommodated state.

This contact occurs during the actuation of the rigid member. Initially, the central portion of the rigid member contacts the anterior surface of the fluid-filled lens. Then as it further actuates, it contacts a larger portion of the lens. Finally it contacts the whole anterior surface of the lens. During this process, the optical properties of the fluid-filled lens are altered initially with a change in only the central portion of the lens, extending radially, and finally throughout the whole optical portion of the lens.

In this manner, the lens may be considered as going from one optical state, S1, to a second optical state S2, with a continuous transition state mechanically spreading through the lens as the rigid component interacts with the lens. The transition state is characterized by a portion of the light focused in optical state S1 and a portion of the light focused in optical state S2. As the transition occurs the percentage of light in the S1 state decreases while the percentage in the S2 state increases.

This type of lens interacts with the natural accommodation mechanism. First, when the eye is focused at far distance, the ciliary muscles are relaxed and the zonules pull tension on the lens capsule. This tension is applied to the rigid component, causing it to come into contact with the full visual field of the fluid-filled lens. As the eye begins to accommodate, the ciliary muscles contract and move inward, releasing tension on the zonules. As this process occurs, the rigid component moves away from the lens, first releasing contact peripherally, and then centrally as it moves anteriorly. This creates a transition state, based on the periphery of the lens with a radius of curvature corresponding to the natural fluid-filled lens, and the central portion corresponding to the rigid member. When the eye muscles are completely focused on near vision, the lens capsule is relaxed, and the rigid component is no longer in contact with the fluid-filled lens. At this point, the power of the fluid-filled lens is dictated by its natural state.

In various embodiments, the fluid-filled lens portion of the IOL includes, consists essentially of, or consists of a thin membrane well that is filled through one or more valves. The valve(s) provide fluidic access allowing for both filling and evacuation of the fluid preoperatively, intraoperatively, or postoperatively. The lens portion may be spheric, aspheric, toric, or other non-spherical shape for improved aberration reduction. It may be constructed of a biocompatible material or polymer (parylene, silicone, silicone derivative such as a phenyl substituted silicone, acrylic, polysulfone, hydrogel, collagen, or other suitable material). In certain embodiments the shell includes, consists essentially of, or consists of multiple materials (e.g., layered fluorosilicone and silicone, parylene deposition into silicone, etc.). The filling fluid may be a biocompatible refractive material; examples of these include but are not limited to: an oil, silicone oil, fluorosilicone, phenyl substituted, silicone oil, perfluorocarbon, aqueous material such as a sugar water, vegetable oil, gel, hydregel, nanocomposite, or electrically active fluid.

The rigid component may be in the shape of a lens that has a minimal power effect on the system. In other embodiments, it may have a non-uniform radius of curvature or points of contact that touch down onto the lens membrane. In yet other embodiments the rigid component may initially come into contact away from the center of the lens. As an example, it may come into contact with the periphery of the lens and then move to the center of the lens.

The rigid member may have optical properties to correct user vision as well. One example would be to have this stiffer material correct for astigmatism by using a toric shape.

The rigid member may be made of a biocompatible material such as a polymer (parylene, silicone, silicone derivative such as a phenyl substituted silicones, acrylic, polysulfone, hydrogel, collagen, or other suitable material). In other embodiments, it may have shape memory properties or heat activated materials, which may cause shape changes once exposed to a heat source (i.e., laser or light-emitting diode) once implanted into the capsular bag. This shape change may be used to adjust base power, astigmatism or other user optical needs that may have been preexisting, caused during surgery, or post-surgery effects (i.e. base power drift), in the preferred embodiment this rigid component can be manipulated (folded, split, etc.) to fit through a small incision in order to reduce the incision size used for surgery. One example of a shape-memory material is a shape-memory alloy (e.g., nitinol) frame embedded in a silicone. Shape change of the shape-memory alloy causes a change the shape of the silicone.

Yet another group of embodiments of the present application relate to IOLs having external components that provide a high degree of accommodation. Two or more haptics, i.e., non-optical, generally peripheral structures that hold the lens in place within the capsular bag inside the eye and transmit force from the eye to the lens. For example, in accordance with various embodiments, the haptics are positioned on the lens such that the changes in the shape of the capsular bag may be directly translated into a shape change of the IOL, thereby enabling the introduction of a desired accommodation to the lens. In addition, embodiments of the invention include additional features on the haptics and/or on the lens such that the distortion of the lens shape alters the optical power of the IOL without degrading the optical qualify of the lens vision zone, e.g., the modulation transfer function (MTF) parameter.

IOLs in accordance with embodiments of the invention generally include or consist essentially of a soft, deformable shell that accommodates one or more filling fluids (i.e., liquids and/or gases) via one or more valves (e.g., patch valves). The valves are typically accessible from an external portion of the lens with a needle or other fluid line for filling. The valves may be self-sealing, e.g., as described in U.S. patent application Ser. No. 14/980,116, filed on Dec. 28, 2015, the entire disclosure of which is incorporated by reference herein.

In accordance with various embodiments of the invention, the IOL interacts with the surrounding lens capsule of the eye both to maintain its position therein and to change optical power (i.e., accommodate). In general, interaction with the anterior and posterior lens capsules causes the lens to be centered and stabilized inside the lens capsule. In addition, the lens capsule can transmit force from the ciliary muscles in the eye to the fluid-filled IOL. For distance vision, the zonules apply radial tension along the equator of the lens capsule. This causes tension of the lens capsule to be translated to the fluid-filled IOL, causing the anterior and posterior surfaces thereof to he flattened and the equator of the lens capsule to be expanded. In this state, the IOL has low optical power corresponding to distance vision. During subsequent accommodation, the ciliary muscles contract, releasing tension on the zonules, allowing the lens to relax against the lens capsule; the radius of curvature of the anterior and posterior surfaces of the liquid-filled IOL is reduced, optical power of the lens is increased, and near vision is provided.

In general, for interaction with the lens capsule, the lens needs to appropriately fill the capsule. In various embodiments, the size (e.g., diameter) of the IOL is over 40% of the lens capsule size, and in certain embodiments over 60% of lens capsule size. In various embodiments, the size of the IOL may be selected, at least in part, by selecting either or both of the size of the lens bag of the IOL and an amount of filling fluid within the IOL. In various embodiments, the IOL interacts with the anterior capsule surface, the posterior capsule surface, or both capsule surfaces during accommodation.

In certain types of liquid-filled IOLs, the lens preferentially expands along the anterior-posterior (A-P) diameter, with the equatorial portions of the lens expanding less. This may be advantageous, as the lens largely maintains its equatorial shape, which may also improve optical function of the lens. However, matching the lens equator diameter with that of the capsular bag may be more challenging. Thus, in various embodiments of the present invention, the accommodative power of the lens capsule may arise not only from expansion and contraction along the A-P diameter, but also along the equatorial diameter of the IOL. Force-transmitting haptics may be utilized to translate force from the capsule to the IOL (e.g., to the lateral surface thereof).

In accordance with embodiments of the invention, force-transmitting haptics not only retain the IOL within the capsule, but also effectively transmit the force from the equator of the lens capsule to the side of the lens. This translation causes local motion of the sidewall of the IOL. However, the center of the IOL typically does not translate in the x and y directions, i.e., orthogonal axes in the radial direction of the lens (axes orthogonal to the optical axis of the lens). In various embodiments, the proportion of the outer x-axis and y-axis of the lens remains relatively constant during the accommodation process, thus preserving the original optical shape of the lens during the accommodation process. For example, if the lens is spherical, it may remain substantially spherical throughout the accommodation.

The force-transmitting haptics in accordance with embodiments of the invention may increase the accommodative amplitude of a fluid-filled IOL. During the accommodated state of the lens, not only does the lens round up, but the equatorial force-transmitting haptic applies a force to the lateral side of the lens, further increasing lens pressure and decreasing lens radius of curvature on the anterior and posterior sides. In the unaccommodated state, the lens capsule applies pressure to the anterior and posterior sides of the lens, flattening it and providing distance vision. In addition, the force-transmitting haptic decreases the force on the lateral side of the IOL, which further decreases pressure and reduces optic power. Further, various embodiments of the invention minimize or substantially eliminate deformation of various portions of the IOL that may result from force applied to the lens periphery by the haptics. For example, embodiments of the invention minimize deformation of the optical regions of the lens and the anterior and posterior peripheral surface portions of the lens.

The fluid-filled IOLs in accordance with embodiments of the invention differ from conventional solid IOLs. For example, the fluid-filled lens is softer and more flexible than a conventional solid lens, and thus IOLs in accordance with embodiments of the invention utilize different amounts of three transmitted from the capsular bag to introduce similar levels of accommodation. In addition, the optical zone of the fluid-filled lens (i.e., where vision correction takes place and through which the patient sees) may be more vulnerable to degradation of optical quality due to, e.g., wrinkling of the balloon-like lens. Haptics in accordance with embodiments of the invention desirably minimize or substantially eliminate such degradation. Finally, the haptics in accordance with embodiments of the invention have material properties compatible with manufacturing processes utilized to create fluid-filled IOLs.

In various embodiments of the invention, the haptics transmit force to the lens by rotating relative to the lens. Since the lens itself is typically centered in the lens capsule and fits conformally therewithin, the haptics may transmit to rotational force to the lens itself without causing lens rotation. Rotation of the haptics may result in a large deformation of the side of the lens, and may thereby result in an increase in pressure in the lens during accommodation. The rotational force may act on a greater portion of the side of the lens. The lens rotation may be limited by the curvature of the haptic or angle of the haptic relative to the lens equator surface, which thereby acts as a stop at a point of maximum desired accommodation.

In various embodiments of the invention, the shape change of the lens caused by the haptic results from an increase in pressure inside the lens. As the haptic moves with the surrounding lens capsule and ciliary muscle movement, it transmits a force to the lens, thereby increasing the pressure inside the lens and increasing the lens power.

In various embodiments of the invention, a less flexible (or even substantially rigid) annulus is present on (e.g., surrounding) the optical surface of the lens. The annulus acts as a boundary for the anterior and/or posterior surface of the lens when the surface is subjected to force from the haptics. The annulus may constrain the portion of the lens membrane within the annulus to deform uniformly in a spherical manner, regardless of the distribution pattern of haptics disposed around the lens, thereby minimizing or substantially preventing astigmatism of the central optical surface during haptic deformation.

In yet another embodiment, the components including those described above may be inserted at different times or successive steps to create a multiple component fluid-filled intraocular lens. These fluid-filled lenses can be implanted pre-filled, or filled through a valve after implantation. When implanted in a pre-filled state, the lenses often require a larger surgical incision to fit the large size of the lens. Larger surgical incisions are problematic and require longer healing times. In addition, these incisions may induce postoperative astigmatism, and therefore lower postoperative visual acuity. Therefore there is a need for a multiple component intraocular lens system that allows one or more components to he implanted sequentially through small surgical incisions.

The separate components may be mechanically coupled, or have a fluid coupling. In certain embodiments of the invention, one or more portions of the lens come into fluidic contact with another component or component's contents during inflation. The components have interlocking portions which engage during filling and an interface, such as a valve, between the two components is activated or opened during the inflation process. Activation may occur from increased pressure between the two components causing a cracking pressure, or by pushing one component into a valve cracking feature. In other embodiments the valve is cracked after inflation by using a remote energy source such as a laser (e.g. Nd:YAG laser, femtosecond laser, picosecond laser, thermal or other optical source).

By breaking down a complex lens into multiple smaller components, the lens may be implanted into the eye through small surgical incisions. In addition, portions of the lens may be removed and/or exchanged without altering other portions of the lens. This technique is also an advantage when piggybacking lenses inside the eye.

By using a modular component-based implant, it is possible to adjust certain portions of the system individually. As an example, the power of a lens may be adjusted without affecting the haptic portion. In other embodiments, the lens may be adjusted by adjusting the haptic portion of the lens. The haptic portion of the lens may be used to translate the lens portion relative to the eye for better centration, move the lens portion in an anterior or posterior direction, or tip/tilt the lens portion for improved optical resolution, it may also be used to rotate the lens, for example, rotating a toric lens for better angular alignment of the lens with the cornea.

A separate component of the lens may be used to restore or maintain the natural lens capsule configuration, to space the lens capsule from the lens component and to prevent local inflammatory or immune reaction from interfering with the lens component of the multiple component IOL. This includes preventing lens epithelial cells from clouding the lens component or interfering with lens actuation as in the case of an accommodating intraocular lens (AIOL).

In certain embodiments of the invention, one portion of the lens is implanted into the capsule to maintain shape. Before, after, or during implantation, the lens capsule may be modified for better postoperative outcomes. Modification may include using a fluid such as hypotoric aqueous solution (e.g. saline, water, dextrose) or cytotoxic solution (local chemotherapy such as methotrexate, etc. . . . ) to eliminate remnant cells in the lens capsule. Other types of modification include removing portions of the lens capsule, while the lens capsule is supported by this surrounding/haptic component of the IOL.

In an aspect, embodiments of the invention feature an intraocular lens implantable into the capsular bag of an eye. The intraocular lens includes or consists essentially of a flexible membrane defining an interior region for accepting a filling fluid and providing an optical correction to vision and, extending from an outer surface of the flexible membrane, a plurality of haptics for retentively engaging surrounding tissue and transmitting force from the capsular bag to the flexible membrane, thereby altering a shape of at least a portion of the flexible membrane and an optical power of the intraocular lens. The haptics do not coincide with (or overlap) an optical axis of the lens.

Embodiments of the invention may include one or more of the following in any of a variety of combinations. One or more, or even all, of the haptics may be elongated and/or curved. One or more, or even all, of the haptics may be shaped as an S or a partial circle (e.g., half-circle). One or more, or even all, of the haptics may be circular. The plurality of haptics may include, consist essentially of, or consist of four or more haptics. The haptics may be distributed around the flexible membrane (e.g., around an equator of the flexible membrane) substantially symmetrically. The haptics may be asymmetrically distributed around the flexible membrane (e.g., around an equator of the flexible membrane). A spacing around the flexible membrane (e.g., around an equator of the flexible membrane) between each pair of the haptics may be approximately equal. The intraocular lens may include a ring disposed around a periphery of the flexible membrane (e.g., around an equator of the flexible membrane). The ring may define a plurality of apertures, each haptic extending through an aperture. The ring may prevent direct transmission of force from the capsular bag to the flexible membrane. The intraocular lens may include a reinforcing pattern disposed on an inner surface and/or the outer surface of the flexible membrane. The reinforcing pattern may be less flexible than the flexible membrane. The reinforcing pattern may be disposed outside an optical zone of the intraocular lens (i.e., disposed outside of the portion of the lens through which vision of the patient typically occurs). The thickness of all or a portion of the reinforcing pattern is greater than a thickness of the flexible membrane. The reinforcing pattern may have a polygonal shape with a plurality of vertices. One or more, or even all, of the haptics may each extend from the flexible membrane at one of the vertices. The reinforcing pattern may be outside the optical axis. The reinforcing pattern may include, consist essentially of, or consist of straight segments that curve under accommodation. The segments may curve away from the optical axis under accommodation. One or more, or even all, of the haptics may each include, consist essentially of, or consist of a hollow tube.

In another aspect, embodiments of the invention feature an intraocular lens implantable into the capsular bag of an eye. The intraocular lens includes or consists essentially of a flexible membrane defining an interior region for accepting a filling fluid and providing an optical correction to vision, and disposed on an outer surface of the flexible membrane, a plurality of haptics for transmitting force from the capsular bag to the flexible membrane, thereby altering a shape of at least a portion of the flexible membrane and an optical power of the intraocular lens. Each haptic is a solid curved segment extending along a portion of the outer surface of the flexible membrane away from an optical axis thereof. The haptics are spaced around the outer surface of the flexible membrane to define gaps therebetween in a relaxed state of the intraocular lens, a size of each gap decreasing in an accommodated state of the intraocular lens.

Embodiments of the invention may include one or more of the following in any of a variety of combinations. The haptics may surround the optical axis of the flexible membrane. The plurality of haptics may include, consist essentially of, or consist of four or more haptics. In the relaxed state of the intraocular lens, the sizes of the gaps may be substantially equal. The gaps may decrease to approximately zero (i.e., the haptics may contact each other) in an accommodated state of the intraocular lens. The haptics may be attached to the flexible membrane by an adhesive.

In yet another aspect, embodiments of the invention feature an intraocular lens implantable into the capsular bag of an eye. The intraocular lens includes or consists essentially of a flexible membrane defining an interior region for accepting a filling fluid and providing an optical correction to vision, an elastic ring surrounding and spaced apart from the flexible membrane, the elastic ring being configured to accept force from the capsular bag and configured to retentively engage surrounding tissue, and extending from the elastic ring to the flexible membrane, a plurality of haptics for transmitting force from the elastic ring to the flexible membrane, thereby altering a shape of at least a portion of the flexible membrane and an optical power of the intraocular lens.

Embodiments of the invention may include one or more of the following in any of a variety of combinations. One or more, or even all, of the haptics may each include, consist essentially of, or consist of a plurality of segments each having a first end connected to the elastic ring and a second end connected to the flexible membrane. One or more, or even all, of the segments may be linear. For one or more, or even, all, of the haptics, a spacing between the first ends of the segments may be larger than a spacing between the second ends of the segments. For one or more, or even all, of the haptics, the second ends of the segments may meet at a common point on the flexible membrane.

In another aspect, embodiments of the invention feature an intraocular lens that includes, consists essentially of, or consists of a membrane defining a central chamber for containing an optical fluid and a spanning member extending between opposed areas of an internal surface of the membrane. When the central chamber is filled, it provides vision correction when implanted in a patient's eye, the central chamber having an optical axis extending through a vision-correcting optical zone of the central chamber. The spanning member resists expansion and/or collapse of the central chamber.

Embodiments of the invention may include one or more of the following in any of a variety of combinations. The spanning member may be elastomeric so as to restrain expansion of the membrane but not collapse thereof. The spanning member may be stiff so as to restrict both collapse and expansion of the membrane. The spanning member may include, consist essentially of, or consist of a spiral spring. At least a portion of the spanning member may extend along an optical axis of the lens. At least a portion of the spanning member may be continuous and solid. At least a portion of the spanning member may be tubular. The lens may have an optical zone. At least a portion of the spanning member may have a diameter larger than a diameter of the optical zone of the lens. The membrane may be at least partially filled with a first optical fluid. The spanning member may be at least partially filled with a second optical fluid different. The first and second optical fluids may be different. The first and second optical fluids may be the same. The membrane may be at least partially filled with an optical fluid, and the spanning member may be permeable to the optical fluid. The spanning member may join the internal surface of the membrane at first and second opposed ends. Each of the ends may have at least one shaped terminal head member with a distal region attached to or integral with the interior surface of the membrane. At least one of the head members may have a terminal surface area sufficiently small relative to a surface area of the interior surface of the membrane to permit the membrane to bulge upon overfilling with an optical fluid. At least one of the head members may have a terminal surface area sufficiently large relative to a surface area of the interior surface of the membrane to resist bulging of the membrane upon overfilling with an optical fluid. At least one end of the spanning member may include, consist essentially of, or consist of a plurality of branches each terminating in a head member with a distal region attached to or integral with the interior surface of the membrane. At least one of the head members may have a substantially symmetric terminal surface. The terminal surface may be round. At least one of the head members may have a substantially asymmetric terminal surface. The terminal surface may include, consist essentially of, or consist of a plurality of radial projections. The exterior surface of the membrane overlying at least one of the head members may have a plurality of radial grooves. The spanning member may be colored. At least a portion of the spanning member may have a color different from a color of the flexible membrane.

In yet another aspect, embodiments of the invention feature a method of correcting a patient's vision. A fluid-fillable and/or fluid-filled deformable lens having an optical axis is installed within the patient's capsular bag following removal of the natural lens therefrom. A rigid member is installed along the optical axis within the patient's capsular bag. Actuation of the rigid member causes it to releasably contact a portion of a surface of the deformable lens and thereby alter an optical power of the deformable lens. The contacted surface has an area dependent on a degree of the actuation.

Embodiments of the invention may include one or more of the following in any of a variety of combinations. At least a portion of the rigid member may be substantially planar. At least a portion of the rigid member may have a thickness larger than a thickness of a membrane of the deformable lens. At least a portion of the rigid member may have optical power. At least a portion of the rigid member may have no optical power. At least a portion of the rigid member may be a segment of a sphere having as radius larger than a radius of the deformable lens. The rigid member may be actuated by far-distance focus of the patient's eye. The rigid member may be anchored to the capsular bag by one or more flexible coupling members. At least a portion of the rigid member may be polymeric. At least a portion of the rigid member may include, consist essentially of, or consist of a shape-memory material (e.g., a shape-memory alloy). At least a portion of the rigid member may have a shape. The portion of the deformable lens in contact with the rigid member may assume the shape of the rigid member. The rigid member may be deformable so that the portion of the deformable lens in contact with the rigid member only partially assumes the shape of the rigid member.

In another aspect, embodiments of the invention feature a combination that includes, consists essentially of, or consists of a focus-altering component and a fluid-fillable and/or fluid-filled deformable lens having an optical axis and sized to fit within a patient's capsular bag. The focus-altering component includes, consists essentially of, or consists of a rigid member having an interaction surface and, joined thereto, a plurality of flexible coupling members configured for anchoring the focus-altering component to the capsular bag so as to permit interaction within the capsular bag between the rigid member and the deformable lens.

Embodiments of the invention may include one or more of the following in any of a variety of combinations. At least a portion of the rigid member may be substantially planar. At least a portion of the rigid member may have a thickness larger than a thickness of a membrane of the deformable lens. At least a portion of the rigid member may have optical power. At least a portion of the rigid member may have no optical power. At least a portion of the rigid member may be a segment of a sphere having a radius larger than a radius of the deformable lens. The coupling members may be configured to permit the interaction in response to far-distance focus of the patient's eye. At least a portion of the rigid member may be polymeric. At least a portion of the rigid member may include, consist essentially of, or consist of a shape-memory material (e.g., a shape-memory alloy). At least to portion of the rigid member may have a shape. The portion of the deformable lens in contact with the rigid member may assume the shape of the rigid member. The rigid member may be deformable so that the portion of the deformable lens in contact with the rigid member only partially assumes the shape of the rigid member.

In yet another aspect, embodiments of the invention feature a combination that includes, consists essentially of, or consists of a retaining structure and a fillable intraocular lens which, when tilled with an optical fluid, has an optical power, an optical axis and a matable feature. The retaining structure includes, consists essentially of, or consists of (i) a central gap portion comprising a matable feature complementary to the matable feature of the lens, whereby mating of the matable features couples the lens to the retaining structure for retention of the lens within the central gap portion, and (ii) peripheral means for stabilizing the retaining structure within the capsular bag of a patient.

Embodiments of the invention may include one or more of the following in any of a variety of combinations. The retaining structure may have a ring configuration. The retaining structure may have as peripheral edge. The stabilizing means may include, consist essentially of, or consist of a plurality of haptics projecting from the peripheral edge of the retaining structure. The retaining structure may be tillable with a fluid. The retaining structure may be at least partially filled with a fluid. The fluid may be a liquid and/or a gas. One of the matable features may include, consist essentially of, or consist of a tab, and the other matable feature may include, consist essentially of, or consist of a recess. The matable features may be roughened or modified surfaces providing a mechanical interface when in contact. The matable features may be frictional surfaces providing a mechanical interface when in contact. The combination may include means for establishing fluid communication between the lens and the retaining structure. The means for establishing fluid communication may include, consist essentially of, or consist of valve portions on the lens and on the retaining structure. The lens may include a plurality of haptic members and may be coupled to the retaining structure via the haptic members. The lens may not be in contact with the retaining structure. The retaining structure may include, consist essentially of, or consist of a plurality of discrete fillable chambers. The retaining structure may include, consist essentially of, or consist of a secondary lens. The combination may include means facilitating alignment of the intraocular lens and the secondary lens.

These and other objects, along with advantages and features of the present invention herein disclosed, will become more apparent through reference to the following description, the accompanying drawings, and the claims. Furthermore, it is to be understood that the features of the various embodiments described herein are not mutually exclusive and may exist in various combinations and permutations. As used herein, the terms “about,” “substantially,” and “approximately” mean ±10% (e.g., by weight or by volume), and in some embodiments, ±5%. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

### BRIEF DESCRIPTION OF THE DRAWINGS

In the drawings, like reference characters generally refer to the same parts throughout the different views. Also, the drawings are not necessarily to scale, with an emphasis instead generally being placed upon illustrating the principles of the invention. In the following description, various embodiments of the present invention are described with reference to the following drawings, in which;

FIG. 1A is a sectional plan view of a fluid-filled intraocular lens having an internal spanning component in accordance with various embodiments of the invention.

FIG. 1B is a sectional plan view illustrating the effect of the spanning component shown in FIG. 1A upon contraction of the ciliary muscles and consequent expansion of the lens in accordance with various embodiments of the invention.

FIG. 2A is a sectional plan view of a fluid-filled intraocular lens having an internal spanning component with a larger attachment surface in accordance with various embodiments of the invention.

FIG. 2B is a sectional schematic of a fluid-filled intraocular lens having an internal spanning component with a diameter larger than the optical zone of the lens in accordance with various embodiments of the invention.

FIG. 3 is a sectional plan view of a fluid-filled intraocular lens having an internal spanning component with a non-unitary attachment surface in accordance with various embodiments of the invention.

FIG. 4A is an elevational view along the optical axis, and FIG. 4B is a sectional view taken along the line 4B-4B, of a fluid-filled intraocular lens in accordance with various embodiments of the invention.

FIG. 5A is an elevational view along the optical axis, and FIG. 5B is a sectional view taken along the line 5B-5B, of another configuration of a fluid-filled intraocular lens in accordance with various embodiments of the invention.

FIG. 6 is an elevational view along the optical axis of the embodiment shown in FIGS. 4A and 4B, but with grooves along the region where the spanning member joins the lens membrane in accordance with various embodiments of the invention.

FIGS. 7A-7D are schematic illustrations of an intraocular lens transitioning from near vision to far vision in accordance with various embodiments of the invention.

FIGS. 8A and 8B are schematic sectional views (with the optical axis in the plane of the figure) of an intraocular lens having a rigid member not coupled to the lens during actuation inside the capsular bag in accordance with various embodiments of the invention.

FIGS. 9A and 9B are schematic sectional views (with the optical axis in the plane of the figure) of an intraocular lens having a rigid member coupled to the lens during actuation inside the capsular bag in accordance with various embodiments of the invention.

FIGS. 10A-10B are schematic views of intraocular lenses with haptics in accordance with embodiments of the invention;

FIGS. 11A-11C are schematic views of intraocular lenses with haptics in accordance with embodiments of the invention;

FIGS. 12A-12D are schematic views of intraocular lenses with haptics and a protective ring in accordance with embodiments of the invention;

FIGS. 13A-13D are schematic views of intraocular lenses with haptics and a reinforcement pattern in accordance with embodiments of the invention;

FIGS. 14A-14D are schematic views of intraocular lenses with partial-ring haptics in accordance with embodiments of the invention; and

FIGS, 15A-15C are schematic views of intraocular lenses with haptics surrounded by an elastic ring in accordance with embodiments of the invention.

FIG. 16A is a schematic illustration of a central fluid-fillable lens and a solid haptic peripheral component of a multiple-component intraocular lens in accordance with embodiments of the invention.

FIG. 16B is a schematic illustration of the fluid-fillable lens and haptic peripheral component of FIG. 16A in assembled form in accordance with embodiments of the invention.

FIGS. 17A-17D are schematic illustrations of a multiple-component intraocular lens having a central fluid-fillable lens and a fluid-fillable haptic peripheral component in accordance with embodiments of the invention.

FIGS. 18A-18C are schematic illustrations of an exemplary coupling mechanism to provide fluidic continuity between two components of an intraocular lens after implantation in accordance with embodiments of the invention.

FIGS. 19A-19C are schematic illustrations of an exemplary coupling mechanism to provide fluidic continuity between two components of an intraocular lens after implantation in accordance with embodiments of the invention.

FIGS. 20A and 20B are schematic illustrations of an intraocular lens with a surrounding haptic component in accordance with embodiments of the invention.

FIGS. 21A and 21B are schematic illustrations of an intraocular lens having haptic components with filling valves connected to a central fluid-fillable lens in accordance with embodiments of the invention.

FIGS. 22A-22C are schematic illustrations of an intraocular lens having a piggyback lens component in accordance with embodiments of the invention.

### DETAILED DESCRIPTION

A. Internal Components

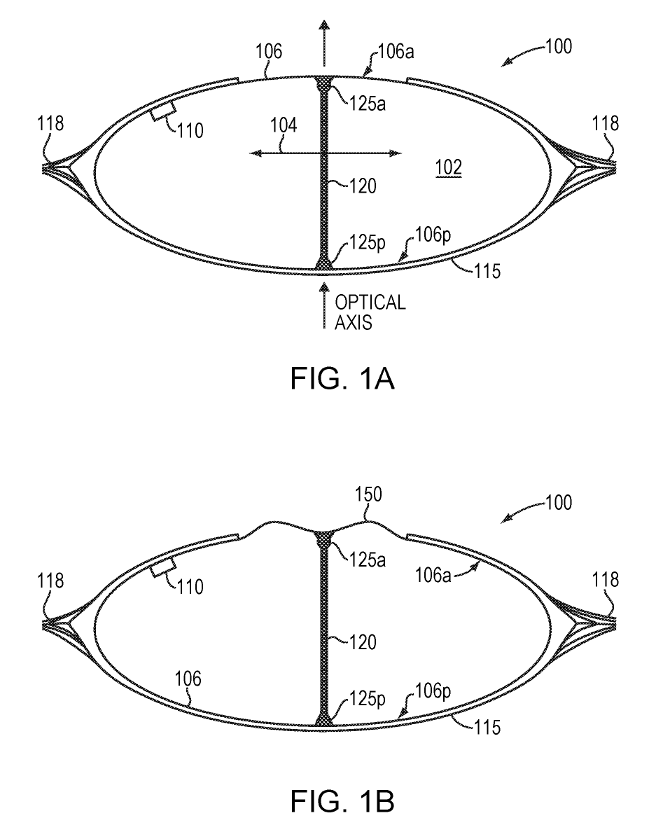

FIG. 1 depicts a liquid-filled accommodating IOL 100 having an interior region 102, which includes an optical zone 104 through which the optical axis passes as indicated. The lens is defined by a membrane 106, which may be made of a flexible polymeric material such as silicone or parylene. The membrane has an anterior side 106a and a posterior side 106p, which faces the patient's retina following implantation. A valve 110 facilitates filling and, in some embodiments, refilling of the AIOL 100 with an optical fluid (e.g., silicone oil). The lens 100 is surrounded by the capsular bag 115, which is itself bound to the zonules 118 on opposed sides. A spanning member 120 extends along the optical axis and joins the interior surface of the membrane 106 at opposed points. One or both of the ends 125a, 125p of the spanning member 120 may be shaped to produce a desired mechanical response as the lens 100 is stretched or released by the zonules 118 and/or is filled with an optical fluid. In some embodiments, the spanning member 120 acts as a string or rope, limiting the expansion of the membrane 106 when the zonules 118 tighten and/or the lens is overfilled. In such embodiments, the spanning member 120 may fabricated from a malleable polymer with adequate tensile strength to avoid breakage and a sufficient Young's modulus to avoid stretching in response to accommodation-induced stress. For example, the spanning member may be polyester, polyethylene, PTFE, silicone, or parylene.

In other embodiments, the spanning member 120 is a spring in tension with a predefined spring constant, k; as a result, the spanning member maintains a length dimension of L with an allowable expansion of ΔL, thereby limiting the amount of curvature change of the anterior and posterior membrane surfaces 106a, 106p. In this case, the spanning member may be an elastomeric polymer. i.e., a polymer exhibiting viscoelasticity and a low Young's modulus. Examples include polyurethanes and polybutadiene, and various other polymers. Alternatively, the spanning member 120 may be a spiral spring.

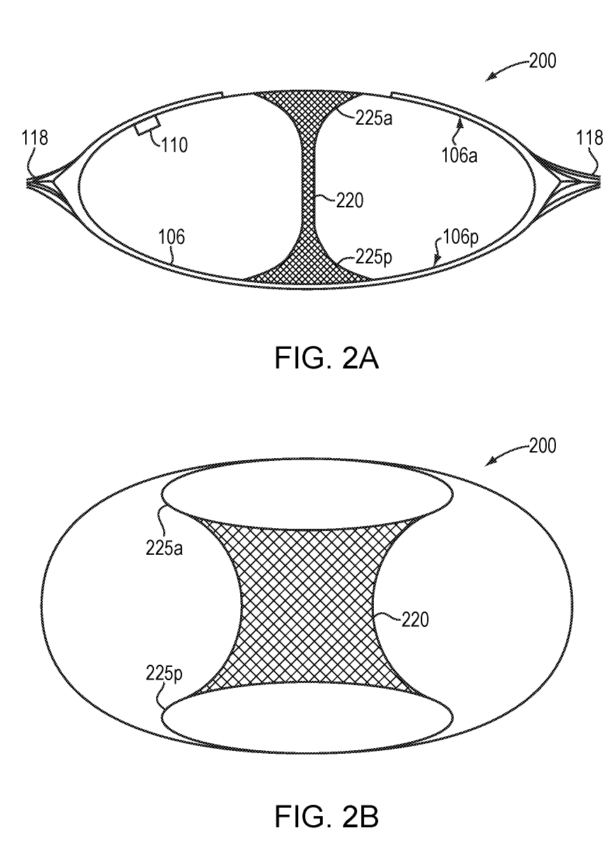

The spanning member 120 is preferably optically clear and/or slender enough to negate any effect on vision. Optical clarity may be obtained by index-matching the spanning member 120 to the fluid filling the lens 100 in order to reduce light scatter and improve optical properties. Alternatively or in addition, the spanning member 120 may be permeable to the filling fluid, with the filling fluid altering the refractive properties of the spanning member 120. For example, the spanning member may be a permeable polymer or shaped as a tube that is filled with an optical fluid (which may have the same or a different refractive index from that of the lens filling fluid). As depicted in FIG. 2B, the tube may, for example, have an internal diameter greater than that of the optical zone of the lens 200. The tube may include one or more apertures to allow the influx and egress of fluid from the interior of the lens 200 into the tube. Alternatively, the tube may include one or more valves that facilitate separate injection and removal of fluid in a fashion similar to that of the lens 200. The fill volume within the tube may be further adjusted to define various curvatures and profiles (e.g., to correct astigmatism and other asymmetric optical anomalies) within the optical zone. The tube desirably contributes no optical aberrations to affect visual acuity.

More generally, all or part of the spanning member 120 may be colored to facilitate visual detection thereof once the lens is implanted, signaling incorrect fill volume and/or the need for fill volume adjustment. The ends 125a, 125p of the spanning member 120 may be joined to the interior surface of the membrane 106 in any suitable manner, e.g., by an adhesive such as epoxy or by heat, or the spanning member 120 may instead be co-molded with the membrane so as to be an integral part of the lens structure. Although the spanning member 120 is shown as a solid structure, in some embodiments it is molded as a spiral spring from a polymer having a desired stiffness.

The profiles of the ends 125a, 125p influence the mechanical response of the lens 100 not only to natural ocular accommodation but also to filling of the lens 100 via the valve 110. With reference to FIG. 1B, when the volume of optical fluid in the lens 100 exceeds its nominal volume, increasing internal pressure causes the anterior and posterior surfaces 106a, 106p to bulge in a manner dependent on the elasticity of the membrane 106, the physical boundary conditions imposed by surrounding tissue structures, and the profile of the ends 125a, 125p. In the case of the lens 100, the ocular anatomy may permit the emergence of a circular anterior bulge 150. In some embodiments, this bulge may be small enough to avoid affecting vision but large enough to increase adhesion to the lens capsule 115; but if the lens membrane 106 is sufficiently yielding, the bulge 150 may contribute to the optical correction provided by the lens 100.

To reduce or avoid the emergence of a bulge, the profile of the spanning-member ends can be altered, as shown in FIG. 2A, to present a larger interface surface. The spanning member 220 has ends 225a, 225p that terminate in broad surfaces covering a larger area of the membrane 106; that is, the surface area of the head member 225a where it joins the inner surface of the anterior membrane portion 106a (by adhesion or co-molding) is sufficiently large relative to the surface area of the membrane portion 106a that, in concert with the surrounding anatomy, the head-member surface precludes or discourages the formation of bulges in the lens membrane 106 resulting from overfilling the lens 200. The width (diameter) of each end 225 is typically no greater than 12 mm, and in some embodiments 5 mm or less. In other embodiments, the surfaces of one or both of the ends 225a, 225p span the entire interior lens surface 106a, 106p, thereby distributing the strain, caused by overfilling over this entire region. It should be understood, however, that the surfaces of one or both of the ends 225a, 225p need not be uniform or symmetric round), or identical, or even unitary. For example, the profile of the end 225a may be optimized experimentally and/or with modeling to deform and minimize unwanted aberration as the lens 200 decreases in optical power during accommodation; as an example, the profile may vary radially in thickness. In other examples, the curvature provides a toric lens profile. But the end 225p may be broad for stress relief or small so as not to interfere with vision. That is, the profile of the end 225a may be dictated by optical considerations and that of the end 225p by mechanical or manufacturing considerations.

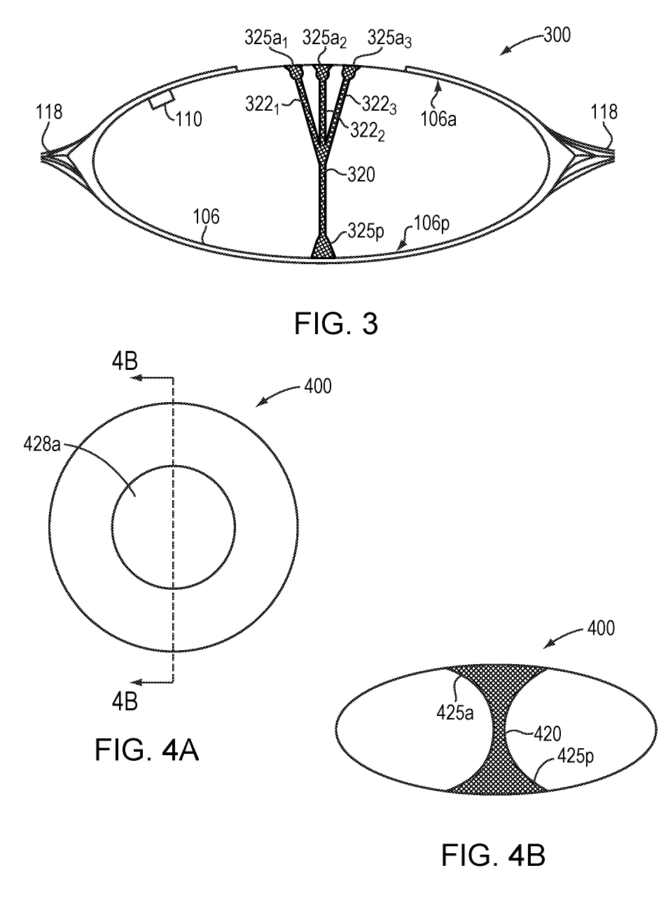

A non-unitary end is illustrated in FIG. 3. Here the spanning member 320 forks into three branches 322₁, 322₂, 322₃ that terminate in ends 325a₁, 325a₂, 325a₃, respectively. Each of the ends 325a may have the same or a different contact area with the interior lens surface 106a. It is the number and distribution pattern of the branches 322 and ends 325, however, that determine the mechanical response of the lens to overfilling and/or accommodation-related stresses. By adjusting the placement of the branches 322 (which may also be present on the posterior side of the spanning member 320), the anterior and posterior membranes 106a, 106p may be customized to better shape the lens 300 to an individual's natural anatomy in addition to fixing the maximum expansion of the lens 300. Multiple branches 322 also distribute the strain on the membrane 106. Multiple branches 322 may be arranged in a line or a small grouping in order, for example, to cause a bulge in the membrane 106 to occur outside the optical field and create an optical anomaly (e.g., different refraction, color change, shading, etc.) that signals an undesirable fill volume.

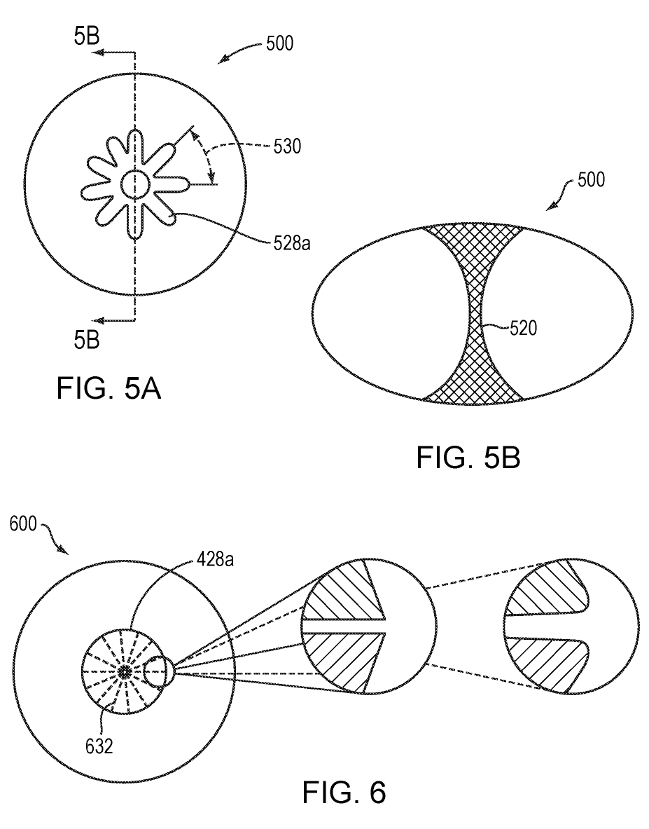

Uniform and non-uniform end interfaces or attachment surfaces are illustrated in FIGS. 4A through 5B. FIGS. 5A and 5B illustrate spanning members 420, 520 that appear, in sectional plan view, similar to the spanning member 220 shown in FIG. 2A. The head-on elevational views of FIGS. 4A and 4B, however, which run along the optical axes of the lenses 400, 500, show different profiles of the attachment surfaces 428a, 528a of the spanning members 420, 520. The symmetric attachment surface 428a is round. The asymmetric attachment surface 528a has a series of finger-like projections emanating from a central point and having a desired angular offset 530 (approximately 40° in the illustrated embodiment).

As shown in FIG. 6, the membrane portion overlying the round attachment surface 428a may have grooves or areas of reduced thickness 632 that run radially from a central point or along a custom path. These discontinuities may start in the optical center of the lens 600, or may begin at a discrete radius from the optical center of the lens. They permit the resulting wedge segments to expand as pressure inside the lens increases, due to, for example, accommodation or overfilling.

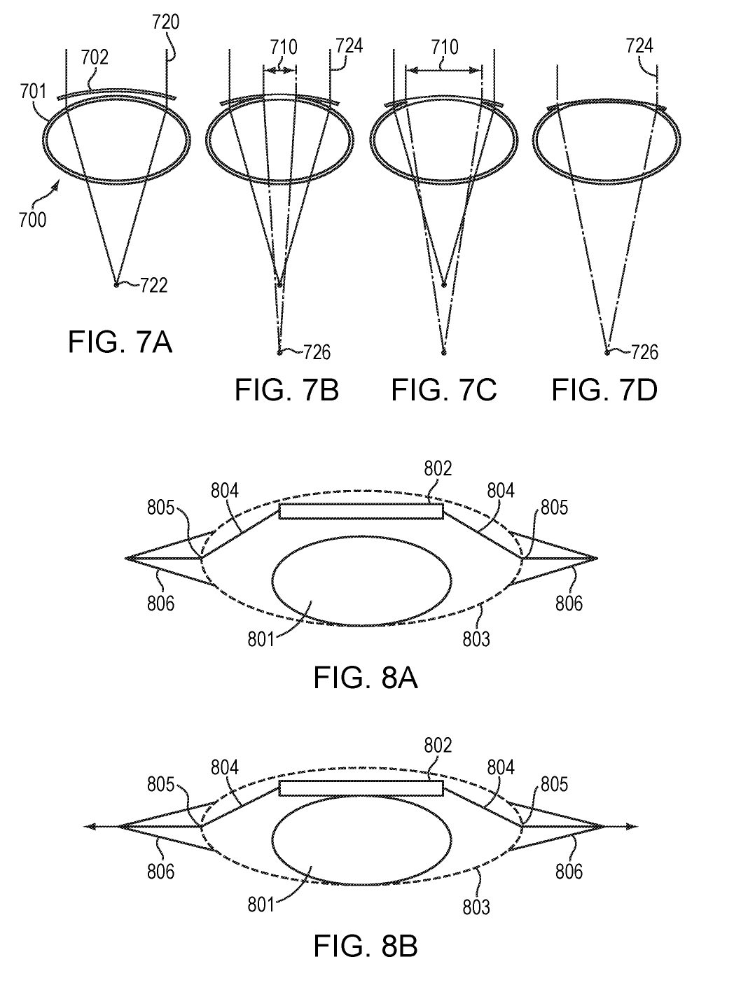

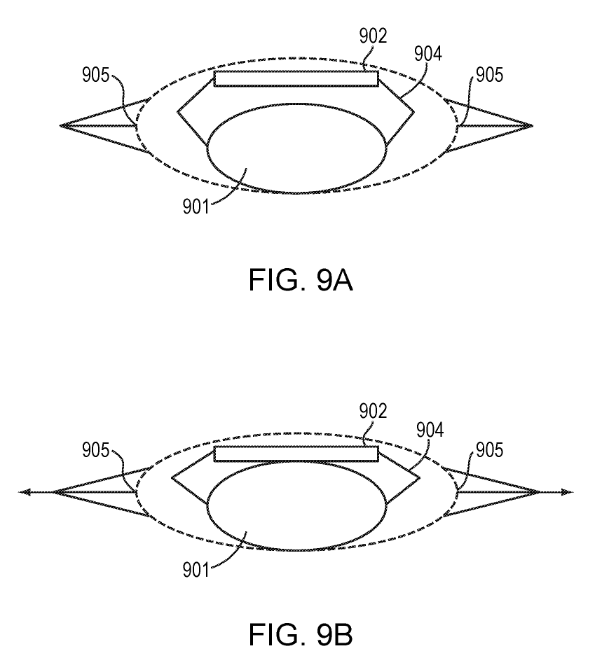

FIGS. 7A-7D depict an IOL 700 as it transitions between near and far vision. IOL 700 comprises or consists of a rigid component 702 and a liquid-fillable lens 701. Rigid component 702 and fluid-filled lens 701 may or may not be mechanically coupled. Light 720 passes through rigid component 702 without significant deviation or modification, as rigid component 702 has minimal optical properties and is substantially transparent. Light then passes through lens 701 and is focused at near point 722. This corresponds to an accommodated lens and 100% of the light is focused at near focal point.

FIG. 7B depicts a substantially planar rigid component 702 as it comes into contact with fluid-filled lens 701. Contact area 710 corresponds to the area where rigid component 702 deforms fluid-filled lens 701, causing the contacting area of the anterior surface to have a different curvature and therefore a different refractive power. The light 724 that is refracted through the rigid component 702 and the deformed portions of fluid-filled lens 701 focuses at point 726, which has a longer focal length than light contacting the peripheral non-deformed portions of the fluid-filled lens. The peripheral portions of fluid-filled lens 701 continue to focus at near point 722. In this intermediate condition, the lens is acting as a multifocal lens, with portions focusing near and other portions focusing far.

FIG. 7C depicts further surface contact between rigid component 702 and fluid-filled lens 701. Contact area 710 continues to expand radially, until it encompasses the entire optical portion of fluid-filled lens 701. FIG. 7D depicts full contact between rigid member 702 and fluid-filled lens 701. In this configuration, 100% of the focused light is focused at far point 726 and there is no multifocality of the lens.

In this manner, this type of lens can act as a variable multifocal lens. In the two extremes (near and for viewing), the lens is a monofocal IOL, with 100% of the light projected for near or far viewing respectively. Intermediate viewing is associated with varying amount multifocality which gradually transitions in percentage from near viewing to far viewing.

FIGS. 8A and 8B depict fluid-filled lens 801 within a patients capsular bag 803. In FIG. 8A, the rigid component 802 is not in contact with the fluid-filled lens 801. In other embodiments, the rigid component may have a skirt or ring-like structure that can slide around the outside of the fluid-filled lens 801. The rigid component 802 may then have corresponding pins or holes in it that guide it vertically up and down during actuation, maintaining alignment between the rigid component and fluid-filled lens 801. The rigid component 602 can be in contact with the capsular bag 803 in some embodiments. In some embodiments this contact is light and does not significantly affect the capsular bag 803, while in other embodiments the rigid component 802 may be pushed anteriorly by the legs 804, causing the capsular bag 803 to expand further vertically and collapse radially. The legs 804 push off the edge of the capsular bag equator 805, where the zonules 806 connect and force the rigid component 802 anteriorly relative to the optical axis.

FIG. 8B depicts the zonules 806 applying a radial force (indicated by the arrow) to the equator 805 of the capsular bag. This tension expands the capsular bag radially and collapses it vertically. The capsular bag 803 in one embodiment compresses to the fluid-filled lens 801 and rigid component 802. Anterior force on the fluid-filled lens 801 causes the posterior membrane of the fluid-filled lens 801 to flatten, which aids in accommodation. Outward expansion of the equator of the capsular bag 805 allows the legs 804 to also extend radially. As the legs 804 extend radially, the rigid component 802 is pushed posteriorly onto the fluid-filled lens 801. This motion is also aided by the compressive force applied by the capsular bag 803 to the anterior surface of rigid component 802. The rigid component 802 is then urged against the fluid-filled lens membrane, causing the lens membrane to change shape. In one embodiment, the rigid component 802 is stiff enough that it deforms the lens membrane to the shape of the rigid component 802. In other embodiments the rigid component 802 has points that protrude out toward the fluid-filled lens 801. These protrusions make contact with critical fulcrum points on the fluid-filled, lens 801. These fulcrum points deform the lens membrane.

FIGS. and 9B depict a fluid-filled lens 901, a rigid component 902 and coupling members 904 that space the fluid-filled lens 901 from the rigid component 902. Coupling members 902 nominally maintain the rigid component anterior to fluid-filled lens 901 and centered to the optical axis as shown in the top view. Coupling members 902 may be hinged as shown.

When the zonules go into tension, as seen as the arrow in the bottom view, the capsular bag 903 expands radially and with a decrease in anterior-posterior (A-P) thickness as shown in the figure. The A-P thickness decrease of the capsular bag 903 causes the bag to compress the posterior membrane of the fluid-filled lens 901 and brings the rigid component 902 into contact with anterior surface of fluid-filled lens 901. In some embodiments, the compressive force on the posterior side of the fluid-filled lens 901 causes the membrane to deform, causing the lens 901 to press on the posterior membrane and undergo an optical power change.

In various embodiments, the rigid component 902 is stiff enough to cause the fluid-filled lens membrane to conform to its shape. In other embodiments, the rigid component 902 has points that protrude out toward the fluid-filled lens 901. These protrusions make contact with critical fulcrum points on the fluid-filled lens 901 (not shown). These fulcrum points then deform the membrane of the fluid-filled lens. In another embodiment, the rigid component 902 has some flexibility to it so that as it contacts fluid-filled lens 901, the final state is an intermediate state between the curvature of the fluid-filled lens and that of the rigid component 902. In this embodiment, the lens acts both as a multifocal lens and an accommodating IOL. In addition, the contact edges between the fluid-filled lens 901 and the rigid component 902 may be less discontinuous, leading to smooth transition between far and near focal points.

In another embodiment of the invention, coupling features 904 interact with the equatorial region of the capsular bag 905. In this embodiment the coupling features 904 maintain contact between the fluid-filled lens 901 and the rigid component 902. The equatorial region of the capsular bag pushes radially on the coupling, members and moves the rigid member away from fluid-filled lens 901.

In certain embodiments, a structure around the fluid-filled lens 901 connects to the fluid-filled lens 901 and the coupling members 904. In other embodiments, the coupling members act as a spring and may be assisted by the expansion and contraction of the capsular bag equator 905. The coupling members may interact with the lens through a series of legs that extend to the lens periphery. In addition, these extensions that extend from the equator to the coupling member may be used to increase leverage or displacement and aid in the movement of the rigid component.

The rigid component may itself have a non-spherical shape, with the possibility for correction or induction of aberration into the lens. In certain configurations it has multifocality itself, thereby convening the lens into a lens with a multifocal surface when engaged. The rigid component itself may also be to lens (monofocal, toric, multifocal, aspheric, and other configurations known to those skilled in the art).

In other configurations, the rigid component deforms itself when engaging with the lens. Instead of two focal lengths, there is a smooth continuous change in curvature of the lens during engagement. In this manner, it may act as a smooth transition between near and far focus, with no multifocality. In this manner, the lens may be considered to have accommodation as well as a shift in multifocality. If the rigid component is flexible enough, the lens may act entirely or almost entirely as an accommodating intraocular lens, with a smooth monofocal transition between near and far viewing distance.

In some embodiments the rigid component may have features that help integrate itself into the capsular bag. In some embodiments small holes could be cut into the capsular bag where small protrusions from the rigid component would stick through. As the capsular bag fibrosis, it will integrate with the protrusions on the rigid component. Other embodiments include but are not limited to: hooks, clasps (around the capsulorhexis), or snap in features (such as a male and female piece with the capsular bag locked inbetween).

B. External Components

Embodiments of the present invention feature fluid-filled (e.g., liquid-filled) accommodating IOLs having one or more haptics for force translation from and retention within the eye capsular bag. In various embodiments, the haptics are attached to the fluid-fillable lens of the IOL during manufacture thereof. In various embodiments, the stiffness (and/or other mechanical properties) of the haptics are selected to enable effective force transmission between the fluid-filled lens and the capsular bag. For example, greater flexibility may result in less force transmission to the lens while less flexibility may result in greater force transmission to the lens. Haptics in accordance with embodiments of the invention may include, consist essentially of, or consist of elongated filaments or fibers having any of a variety of different shapes. The material of the haptic may be different from that of the lens bag of the IOL and attached thereto during the manufacturing process.

In various embodiments, the lens haptics include, consist essentially of, or consist of one or more biocompatible materials such as acrylic, polypropylene, polyvinylidene fluoride (e.g., KYNAR.), polyethersulfone, silicone, polyester, parylene, and/or a shape-memory alloy (e.g., an alloy of nickel and titanium such as nitinol). In various embodiments, the lens haptics may be composed of one or more non-biocompatible materials that are coated with one or more biocompatible materials. In various embodiments, the haptic includes, consists essentially of, or consists of a solid fiber or hollow tube of one or more materials that may be encapsulated by a coating of one or more different materials, e.g., to select a desired stiffness of the haptic. The thickness and/or composition of such coatings may be varied in different portions and/or along the length of the haptic in order to locally vary the flexibility of one or more portions of the haptic.

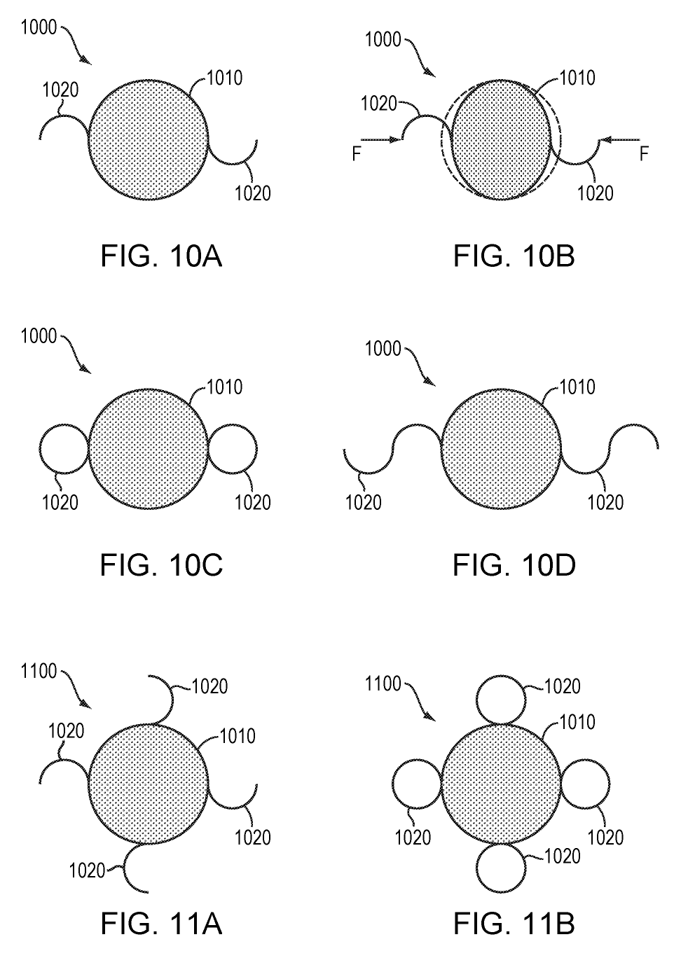

In various embodiments, IOLs each have only two haptics for force transmission from the capsular bag to the lens. For example, the two haptics may be oriented directly across from each other along a diameter (e.g., the equatorial diameter that is perpendicular to the optical axis) of the lens. FIG. 10A depicts a fluid-filled IOL 1000 that includes or consists essentially of a hollow flexible lens 1010 and two haptics 1020 for force transmission from the eye capsular bag to the lens 1010. In various embodiments, each haptic 1020 includes, consists essentially of, or consists of an acrylic fiber having a half-circle shape and that is attached to the lens 1010 with, e.g., a silicone adhesive and projects therefrom like a hook. IOL 1000 is depicted in a relaxed state in FIG. 10A, in FIG. 10B, the IOL 1000 is depicted in an accommodating state in which a compressive force F is applied to the haptics 1020, deforming the lens 1010. As shown, the shape of the lens 1010 is altered by the force F compared to its original, relaxed state (depicted as a dashed outline).

Haptics in accordance with embodiments of the invention may have any of a variety of shapes different from the half-circular shape shown in FIGS. 10A and 10B. FIG. 10C depicts an IOL 1000 having two circular haptics 1020. For a given haptic material and fiber diameter, the full-circular shape of the haptics 1020 in FIG. 10C will generally be more rigid than the half-circular haptics shown in FIG. 10A and 10B and therefore may more efficiently translate compressive force from the eye capsular bag to the lens 1010. In some embodiments, the haptics may be three-dimensional (e.g., spherical) rather than two-dimensional. FIG. 10D illustrates an IOL 1000 having two S-shaped haptics 1020, which may be more flexible than half-circular haptics in various embodiments of the invention and exhibit greater mechanical interaction with the surrounding ocular anatomy. Other shapes for haptics 1020 may be selected by one of skill in the art and are within the scope of the present invention.

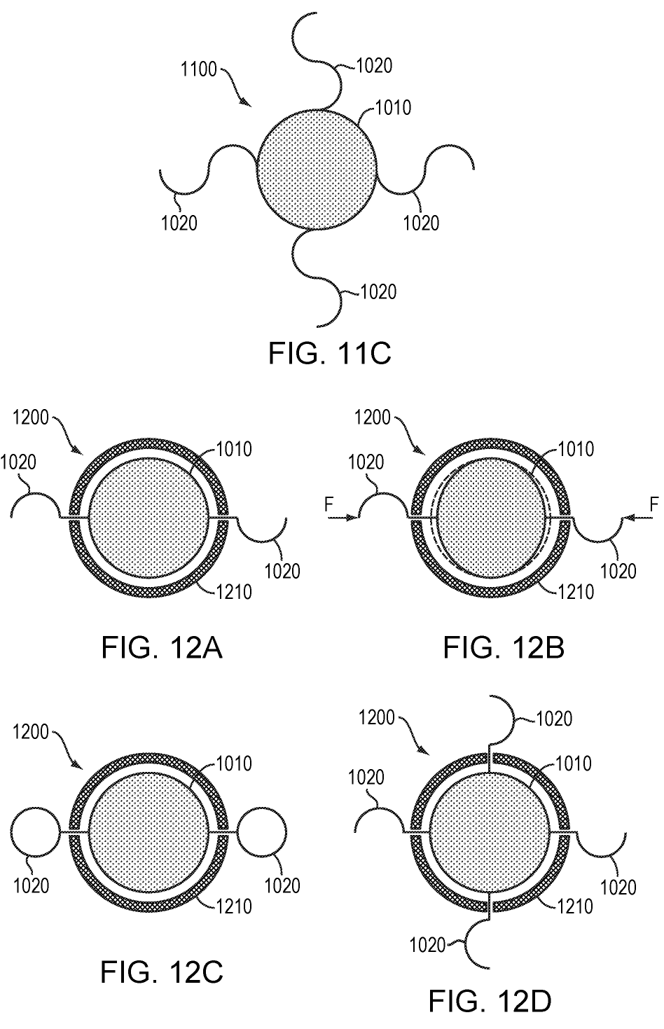

In various embodiments, IOLs may each have three, four, five, or more haptics for force transmission; such embodiments may enable the transmitted flame to be more uniformly distributed around the periphery of the lens. For example, FIG. 11A depicts a fluid-filled IOL 1100 having four half-circular haptics 1020 attached to and spaced approximately equally around the lens 1010. FIG. 11B depicts an IOL 1100 having four circular haptics 1020, and FIG. 11C depicts an IOL 1100 having four S-shaped haptics 1020. Such embodiments may improve the optical quality of lens 1010 via more uniform distribution of the force from the capsular bag.

As mentioned above, the balloon-like lenses of IOLs in accordance with embodiments of the invention are flexible and therefore more vulnerable to degradation of optical quality from, e.g., wrinkling of the lens surface, asymmetric bulging of the optical zone of the lens, etc. Thus, embodiments of the present invention advantageously prevent deformation of the fluid-filled lens of the IOL resulting from direct interaction between the lens and the capsular bag and constrain deformation of the lens to result only (or substantially only) via the haptics attached to the lens. For example, FIG. 12A depicts a fluid-filled IOL 1200 having a solid protective ring or band 1210 disposed around the flexible lens 1010 along a direction that does not pass through the optical axis of the lens; in the figure, the optical axis passes through the center of the ring 1210. As shown, the ring 1210 defines an opening or aperture therethrough for each of the haptics 120 to extend from the lens 1010 to the capsular bag. In this manner, force from the capsular bag is transmitted to the lens 1010 only via the haptics 1020, rather than via any direct interaction between the lens 1010 and the capsular bag. Moreover, the openings in the ring 1210 may also help to constrain motion and/or deformation of the haptics 1020 themselves that might result from compressive force from the capsular bag. FIG. 12B depicts the IOL 1200 in an accommodative state (as contrasted with the relaxed state depicted in FIG. 12A), showing deformation of the lens 1010 resulting from force F transmitted via the haptics 1020 through the ring 1210, IOL 1200 may feature haptics having different shapes—FIG. 12C depicts an IOL 1200 having circular haptics. In addition, IOL 1200 may feature more than two haptics 1020. For example, FIG. 12D depicts an IOL 1200 having four half-circular haptics 1020 each extending through ring 1210 and once again, not passing through the optical axis of the lens. In general, the haptics are arranged symmetrically around the IOL 1200. In some embodiments, however, the haptics are arranged in a manner responsive to the forces of accommodation, e.g., they may be concentrated where the zonules apply maximum force to the IOL.

The ring 1210 may include, consist essentially of, or consist of one or more biocompatible materials such as high-durometer silicone, parylene, acrylic, or collagen or a collagen derivative. As described above regarding haptic 1020, the ring 1210 may be composed of a non-biocompatible material coated with one or more biocompatible materials. According to the material selection of the ring, haptic, and lens, the components may be bonded using an adhesive, overmolded in portions, molded as anterior and posterior pieces, or molded in a unitary piece. In various embodiments featuring ring 1210, one or more of the haptics 1020 may include stops that limit the penetration depth of the haptic 1020 into the interior of ring 1210. In various embodiments, the ring 1210 includes, consists essentially of, or consists of silicone having a cross section of approximately 1 mm×2 mm the ring 1210 is therefore much less flexible than the lens 1010, which may have a thickness of, liar example approximately 20 μm to approximately 100 μm.

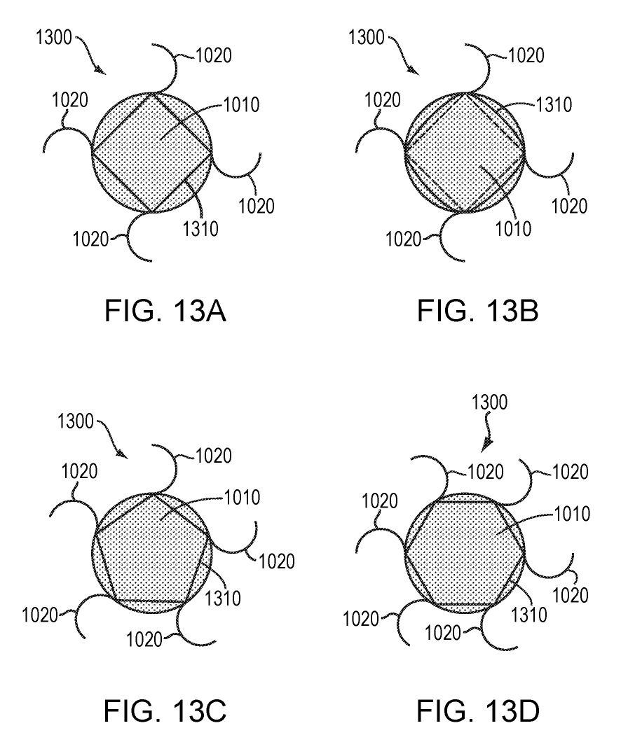

In various embodiments of the present invention, the local elastic properties of the flexible lens of the IOL are altered via incorporation of a reinforcement pattern disposed on the lens surface or within the lens (e.g., at or near the lens equator), ideally outside the optical zone of the lens. Advantageously, the force transmission by the haptics to the lens may be focused at particular portions of the reinforcement pattern and transmitted to the lens through the reinforcement pattern, thereby minimizing or substantially eliminating undesired wrinkling or bulging of other portions of the lens. For example, the reinforcement pattern may have a polygonal shape (e.g., triangle, square, pentagon, hexagon, etc.), with each haptic of the IOL attached to the lens at a point corresponding to one of the vertices of the polygon. For example, FIG. 13A depicts, in a relaxed state, as fluid-filled IOL 1300 having four haptics 1020 that are affixed to lens 1010 at locations corresponding to vertices of a square-shaped reinforcement pattern 1310. The reinforcement pattern is more rigid (i.e., less flexible) than the membrane of lens 1010 and acts as a skeleton or frame to sustain the shape change of lens 1010. FIG. 13B depicts IOL 1300 in an accommodating state, in which compressive force is applied by the capsular bag and transmitted to the reinforcement pattern 1310 via the haptics 1020. As shown, the reinforcement pattern 1310 curves, deforms, and/or stretches into a more circular pattern, thereby altering the shape of the lens 1010 to the shape corresponding to the desired optical power (the original shape of reinforcement pattern 1310 is shown in dashed lines). FIG. 13C and 13D depict IOLs 1300 having a pentagonal reinforcement patter 1310 and a hexagonal reinforcement pattern, respectively. Such higher-order polygons may, in various embodiments, distribute the force transmitted by the haptics more uniformly to the surface of lens 1010. Polygonal reinforcement patterns 1310 having more than six vertices are within the scope of the present invention. Although FIGS. 13A-13D depict a haptic 1020 connecting to the lens 1010 at every vertex of reinforcement pattern 1310, this is not a requirement, and in various embodiments of the invention one or more vertices of reinforcement pattern 1310 are disposed at points on lens 1010 where haptics 1020 do not connect thereto.

As mentioned above, in various embodiments of the invention the reinforcement pattern 1310 is less flexible than the membrane of the lens 1010. For example, the reinforcement pattern 1310 may include, consist essentially of, or consist of a less flexible material than the membrane, and/or may have a lamer thickness than that of the membrane. The reinforcement pattern 1310 may include, consist essentially of, or consist of, for example, a biocompatible material such as silicone, a silicone derivative such as a fluorosilicone, phenyl-silicone, or parylene. The reinforcement pattern 1310 may be fabricated on the lens 1010 membrane via, for example, local deposition (e.g., vapor deposition), molding, or a coating process such as spray- or dip-coating. In various embodiments, the reinforcement pattern 1310 is composed of a coating that is a dispersant with a volatile component and a non-volatile component. In such embodiments, the dispersant has a low viscosity to allow coating and/or shaping until the volatile component is evaporated from the base material (e.g., a polymer).

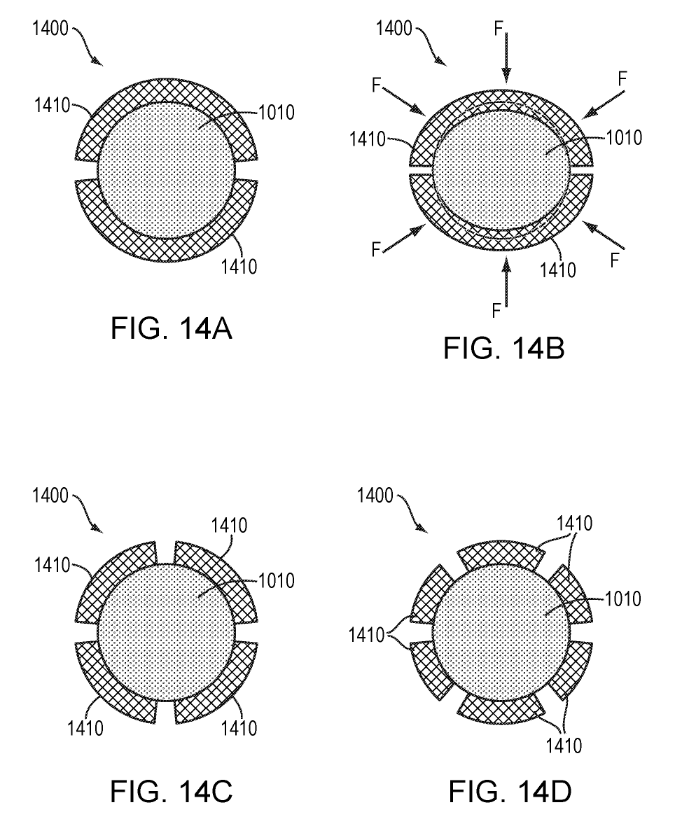

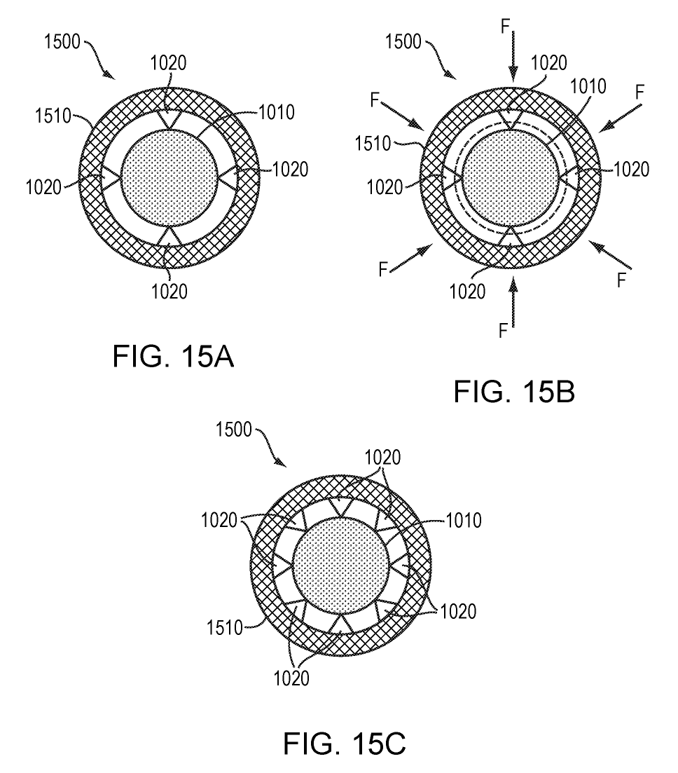

The haptics of the IOL need not be elongated fibers, the ends of which are affixed to the lens at a point. Rather, in accordance with embodiments of the present invention, haptics may include, consist essentially of, or consist of partial curved rings that each surrounds a portion of the periphery of the lens. In such embodiments, the IOL may feature two or more haptics that collectively contact and surround only a portion of the periphery of the lens—gaps between the partial-ring haptics allow the lens to change shape in response to the force transmitted to the lens by the haptics. The partial-ring haptics may be substantially rigid rather than flexible and thus not deform while transmitting force from the capsular bag to the lens of the IOL. (In other embodiments, the partial-ring haptics may be flexible but preferably less flexible than the membrane of the lens.) As an example, FIG. 14A depicts a fluid-filled IOL 1400 featuring two partial-ring haptics 1410. As shown, the haptics 1410 partially surround, while defining gaps along, the periphery of lens 1010. FIG. 14B depicts the IOL 1400 in an accommodative state under a force F from the capsular bag. As shown, the lens 1010 is deformed from its original shape (shown as a dashed line) as the haptics 1410 compress the lens 1010 toward each other, at least partially closing the gaps between the haptics. In various embodiments, the gap distance between the haptics corresponds to the maximum amount of deformation of the lens 1010 that the lens can tolerate (for example, before rupture or irreversible shape change) and/or to a maximum amount of optical power change desired in the patient's eye; once the gaps between the haptics 1410 close and the haptics 1410 come into contact, the haptics 1410 may be sufficiently rigid such that no additional deformation of the lens 1010 occurs. In various embodiments of the invention, an IOL 1400 may feature more than two partial-ring haptics 1410, as shown in FIG. 14C (four partial-ring haptics 1410) and FIG. 14D (six partial-ring haptics 1410). In various embodiments, the greater the number of partial-ring haptics 1410 utilized to transmit three from the capsular bag, the more uniform is the resulting deformation of the surface of the lens 1010.

Partial-ring haptics 1410 may include, consist essentially of, or consist of one or more biocompatible materials such as acrylic, polypropylene, polyvinylidene fluoride (e.g., KYNAR), polyethersulfone, silicone, polyester, parylene, shape-memory alloy (e.g., an alloy of nickel and titanium such as nitinol). In various embodiments, the haptics 1410 may be composed of one or more non-biocompatible materials that are coated with one or more biocompatible materials. The haptics 1410 may be pre-manufactured and attached to the lens 1010 via, e.g., an adhesive (e.g., silicone adhesive), or the haptics 1410 may be molded together with the lens 1010 during fabrication thereof. In various embodiments, the haptics 1410 may be deposited (e.g., vapor deposited) on the lens 1010 or spray- or dip-coated onto the lens 1010.

In various embodiments of the present invention, the haptics do not transmit force directly from the capsular bag to the lens of the IOL. Instead, a flexible, elastic ring may surround the periphery of the lens and be connected to the lens is two or more haptics. (As used herein, the term “ring” is used to connote a closed shape that is not necessarily circular; rather, a ring. may be, e.g., elliptical, polygonal, or irregular in shape.) In such embodiments, the force from the capsular bag first distorts the flexible ring, which in turn deforms and/or translates the haptics, resulting in deformation of the shape of the lens. In some embodiments, the haptics may extend partially or completely through apertures defined by the flexible ring, similar to the configuration described above for IOL 1200 (and, in such embodiments, the haptics and/or the ring may incorporate stops to retard or prevent further motion of the haptics after a pre-determined amount of force is transmitted thereby). FIG. 15A depicts an exemplary fluid-filled IOL 1500, in accordance with embodiments of the invention, which includes an elastic ring 1510 surrounding and in contact with multiple haptics 1020, which in turn are affixed to the lens 1010. As shown, the haptics may be have a V shape (or may be individual linear haptics assembled into multiple V shapes). While FIG. 15A depicts IOL 1500 in a relaxed state, FIG, 5B shows IOL 1500 in an accommodated state in which a force F has compressed the ring 1510, in turn compressing the haptics 1020, which alter the shape of the lens 1010. While FIG. 15A and 15B show IOL 1500 having four haptics 1020, embodiments of the invention may have fewer or more than four haptics 1020 surrounded by ring 1510. For example, FIG. 15C depicts IOL 1500 as having eight haptics 1020.

The ring 1510 may include, consist essentially of, or consist of one or more biocompatible materials such as acrylic, polypropylene, polyvinylidene fluoride (e.g., KYNAR), polyethersulfone, silicone, polyester, parylene, shape-memory alloy (e.g., an alloy of nickel and titanium such as nitinol). In various embodiments, the ring 1510 may be composed of one or more non-biocompatible materials that are coated with one or more biocompatible materials. The ring 1510 may be pre-manufactured and attached to the haptics 1020 via, e.g., an adhesive (e.g., silicone adhesive), or the ring 610 may be molded together with the haptics 1020 and/or the lens 1010 during fabrication thereof.

IOLs in accordance with embodiments of the invention may be implanted with minimal or no volume of fluid within the lens to decrease IOL size and this the incision size required to implant the IOL within a patient's eye. The lens may contain one or more valves accessible from an external portion of the lens with a needle or other fluid line for filling. Such valves may be self-sealing, e.g., as described in U.S. patent application Ser. No. 14/980,116, filed on Dec. 28, 2015, the entire disclosure of which is incorporated by reference herein.

C. Multiple-Component IOL

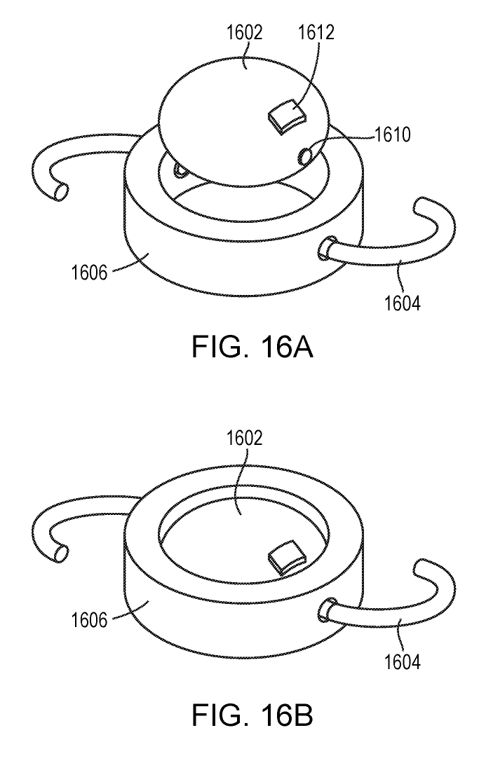

Refer to FIGS. 16A and 16B, which depict a multiple-component IOL with a central liquid-filled lens 1602 and a solid haptic peripheral component surrounding the lens 1602. FIG. 16B provides an exploded view, while FIG. 16B illustrates the implantable configuration. The peripheral component comprises or consists of a retaining structure 1606, which may feature two or more projecting haptics 1604. When haptics are used, they are typically attached to retaining structure 1606, which provides the mechanical interface between the haptics 1604 and the liquid-filled lens 1602. Fluid-filled lens 1602 has a valve 1612, which is used to fill the lens 1602. In an embodiment, the retaining portion 1606 is implanted first. Then the fluid-filled lens 1602 is implanted in an empty state, and subsequently filled through valve 1612. During filling, an interface feature 1610 of the fluid-filled lens 1602 comes into contact with the inner surface of the retaining portion 1606 or, in some cases, an end of a haptic 1604. This latter mechanical coupling option allows the haptics 1604 to directly apply force to or retain fluid-filled lens 1602. There may be one interface feature 1612 for each haptic 1604 of the lens assembly.

Haptics 1604 may be free to move radially within the retaining structure 1606, but may have stops that limit total travel in one or more directions. This prevents the haptics from becoming disengaged from the retaining structure 1606 during implantation, from being too far internally to interact with fluid-filled lens 1602, or interfering with fluid-filled lens implantation into the retaining structure. In a similar manner, haptics 1604 may be constrained by the retaining structure 1606 so they do not rotate.

In other embodiments of the invention, haptics 1604 are mechanically constrained and fixed in retaining structure 1606 and provide no mechanical coupling to fluid-filled lens 1602. In such cases, fluid-filled lens 1602 interfaces with the retaining structure 1606 in order to maintain its position relative to the lens capsule. If haptics 1604 are omitted, retaining structure 1606 makes contact with the lens capsule on one or more suffices (e.g., anterior, posterior, peripheral) thereof.

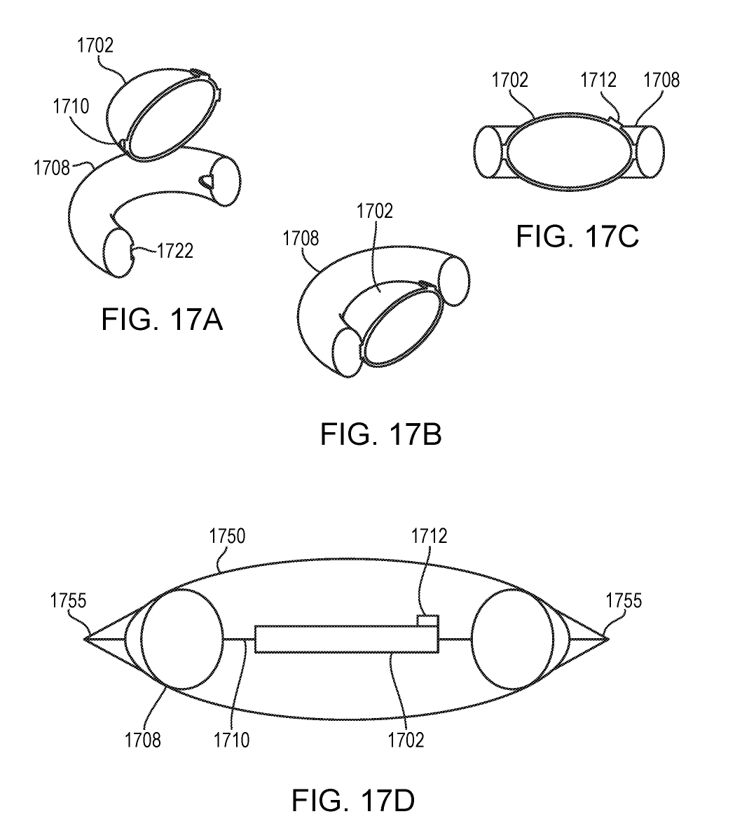

FIGS. 17A-17D illustrate a multiple-component IOL comprising or consisting of a central fluid-filled lens 1702 and a fluid-filled haptic component 1708. FIG. 17A shows the lens 1702 and haptic 1708 in an exploded view and FIGS. 17B and 17C are cutaway isometric and elevational views, respectively. Fluid-filled lens component 1702 has a valve 1712 for filling, interface features 1710 that mate with fluid-filled haptic component 1708 via mating interface features 1722. In this configuration the haptic component 1708 may be implanted first, either filled or unfilled. Next, the fluid-filled lens component 1702 is implanted. During implantation, interface features 1710 mate with interface features 1722 in fluid-filled haptic 1708. As the lens 1702 is filled, the features 1710, 1722 come into mechanical contact and couple. During this process, fluidic continuity between haptic component 1708 and fluid-filled lens component 1702 may be established, and the fluid-filled haptic 1708 is subsequently filled.

In other embodiments, fluid-filled haptic component 1708 is separately filled after implantation, or pre-filled during implantation. In such circumstances, the features 1710, 1722 mechanically restrain and couple the fluid-filled lens component 1702 to the fluid-filled haptic component 1708.

Although shown as discrete elements, interface features 1702, 1722 may be as simple as a radial mechanical interface (e.g., a raised off-round tab and a complementary recess) between fluid-filled lens component 1702 and fluid-filled haptic component 1708 during filling, or may instead be a roughened surface or simple stiction between the two components. This mechanical interface may be enhanced through the use of surface modification (e.g., oxygen and/or nitrogen plasma treatment, parylene deposition into the surface, or adding other functional groups), surface roughness (e.g., etching the surface), or using localized hydrogen bonding, ionic bonding, or hydrophobic bonding between the surfaces. In other embodiments, surface linking is increased by using polymers that continue to cure after implantation. This includes silicone elastomers that have been partially cured, but continue to cure post-implantation.

FIG. 17D shows how, after implantation, the fluid-filled haptic component 208 may wrap at least partially circumferentially around the equator of the capsular bag 1750. The lens component 1702 is attached to the fluid-filled haptic by interface features 1710 or portions thereof. The fluid-filled haptic component 1708 centers both radial and tilt characteristics of the attached lens component 1702 (as shown in the dashed lines) inside the capsular bag 1750. These self-centering and self-alignment characteristics of the haptic component 1708 may be adjusted by modifying the capsule interfacing contours of the haptic component. The fluid-filled haptic component 1708 is located near the anatomical region where the zonules 1755 connect with the capsular bag 1750. The zonules 1755 relax and tighten to change the tension and shape of the capsular bag 1750. As the zonules tighten, the capsular bag 1750 extends radially (i.e., horizontally in FIG. 17D), but also collapses axially (i.e., vertically in FIG. 17D). The positioning of one or more fluid-filled haptic components near the equator of the capsular bag 1750 allows it to transmit the forces and pressure from the zonules efficiently. In some embodiments, the forces may be optionally transmitted via physical contact or fluid interaction between the fluid-filled lens component 1702 and the fluid-filled haptic component 1708.

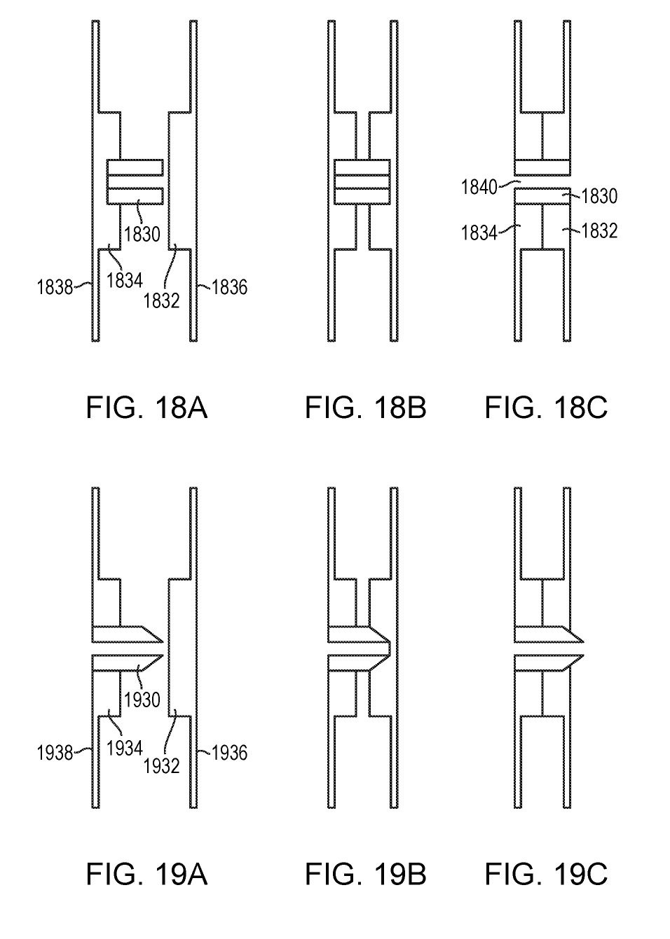

FIGS. 18A-18C depict an exemplary a coupling mechanism to provide fluidic continuity between two components of the IOL after implantation. FIG. 18A shows the components before engagement, FIG. 18B shows partial engagement, and FIG. 18C depicts full engagement of the two components and consequent fluidic continuity between the two components.

One component comprises or consists of a wall 1838 and a valve 1834. The first component has an internal fluid compartment to the left of wall 1838. The second component comprises or consists of a wall 1836 and valve 1832. The internal fluid compartment of the second component is to the right of wall 1836. In FIG. 18A, a penetrating member 1830 is partially engaged in valve 1834 and not engaged in second component valve 1832. As the two components are brought together, penetrating member 1830 engages valve 1832, and then penetrates both valves 1834 and 1832 to allow fluidic contact 1840 between the two components.

FIGS. 19A-19C depict another exemplary coupling mechanism to provide fluidic continuity between two components of the IOL after implantation. Here the first component has a sharp penetrating member 1930, which is integrated with (e.g., co-molded with or permanently attached to) the wall 1938. The internal fluid compartment of the second component is to the right of wall 1936. Contact between the two components causes sharp penetrating member 1930 to penetrate coupling portion 1932 of the second component. This may occur during inflation of either the first or second component.

For example, a haptic component may comprise a haptic wall 1938 and a coupling portion 1934 with a protruding sharp penetrating member 1930. The sharp penetrating member 1930 is already in fluidic contact with the haptic component. When fluid-filled lens component is filled, a lens coupling member 1932 comes into contact with sharp penetrating membrane 1930. As inflation continues, sharp penetrating member 1930 penetrates fluid-filled lens coupling member 1932, leading to fluidic continuity between the haptic and the lens. The haptic component can then be filled along with the fluid-filled lens component.

Other coupling mechanisms are possible. One example uses two-piece valves that couple together and open after interlocking. A second example uses pressure between the lens component and haptic component to seal. A third example uses glue or adhesive that holds the two components together. In certain embodiments, the two pieces come into contact. Then at a later time an aperture is opened between the two membranes using an optical or thermal source, e.g., a Nd:YAG laser, femtosecond laser, picosecond laser, or other thermal or optical source.

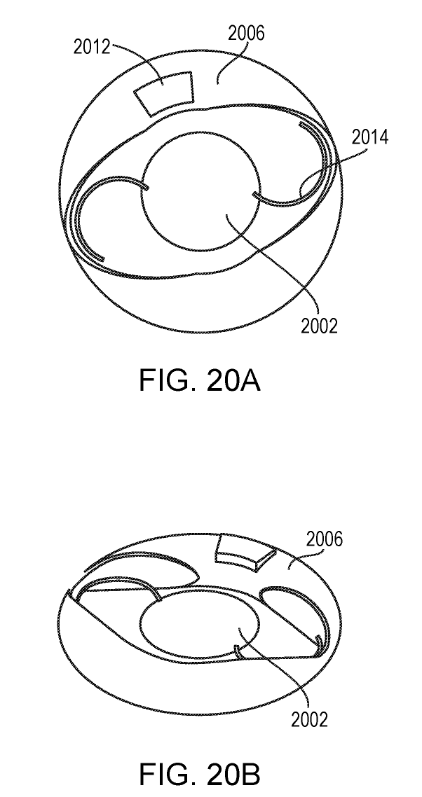

FIGS. 20A and 20B depict an intraocular lens component 2002 with a surrounding haptic component 2006. Haptic component 2006 has a valve 2012, which is used to fill the haptic component. Haptic component 2006 is used to seat intraocular lens component 2002 properly in the lens capsule. It may be implanted, before, during, or after intraocular lens component 2006 has been implanted.

Haptic component 2006 controls the environment around intraocular lens 2014. The environment may determine, for example, specific optical properties, chronic dopants, and pressure that collectively create a net optical outcome in conjunction with the optical properties of the lens component 2002. In addition, haptic component 2006 can be used to adjust the position of the intraocular lens component 2002.

Haptic component can be inflated to space the surrounding lens capsule away from intraocular lens component 2002. In certain embodiments, haptic component 2006 is inserted and inflated, stabilizing the lens capsule. After implantation of haptic component 2006, the lens capsule is modified for better postoperative outcomes. This modification may involve elimination of residual cells and/or lens matrix, or removal of portions of the lens capsule. Cytotoxic agents or agents to prevent chemotaxis of residual lens epithelial cells may be used to prevent cell migration and subsequent capsular opacification and/or fibrosis. Cytotoxic agents include fluids such as hypotoric aqueous solution (e.g., saline, water, dextrose, or mannitol) or cytotoxic solution (e.g., local chemotherapeutics such as methotrexate, etc.). Alternatively or in addition, surface modification (oxygen plasma, ammonia plasma, nitrogen plasma, parylene deposition, etc.) may be used to eliminate remnant cells in the lens capsule. These agents may be applied to the capsule as a lavage, or impregnated into the surface or filling fluid of the lens and/or haptic. Other types of modification include removing portions of the lens capsule while the lens capsule is supported by this surrounding/haptic component of the IOL. For example, after implantation of the haptic member, the posterior lens capsule may be mechanically removed, treated with laser (Nd:YAG, femtosecond laser, etc.), or thermally ablated. After treatment, intraocular lens component 2002 can be implanted into the lens capsule.

The intraocular lens may be positioned within the capsular bag by altering the fluid fill of the haptic component 2006. For example, if intraocular lens component 2002 is mechanically coupled to haptic component 2006, then by increasing the fill in different portions or compartments (not shown) of haptic component 2006, the lens can be repositioned, re-centered, tip/tilted, moved anteriorly or posteriorly. In addition, the lens can be rotated. Therefore, an intraocular lens component 2002 that is already mounted can be optimized either during or post-implantation for better refractive outcomes.

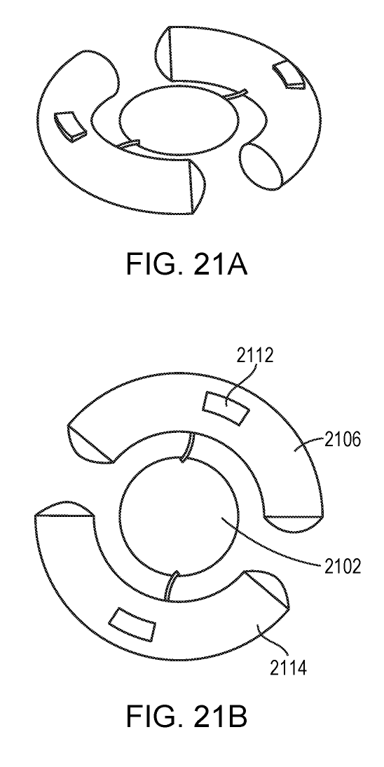

FIGS. 21A and 21B depict a multiple-component haptic member 2106 with one or more filling valves 2112 and an intraocular lens component 2102. This configuration may be used in the same manner as described above with reference to FIGS. 20A and 20B. However, here multiple chambers are depicted, making it more evident how tip/tilting or positioning of the lens occurs with preferential filling of one of the two haptics. In a similar manner, there may be more than two haptic components 2106 (e.g., haptic member 2114), there may be struts mechanically constraining the haptic members 2106, or one or more haptic members may be continuous with multiple filling chambers to allow differential movement of intraocular lens 2102.

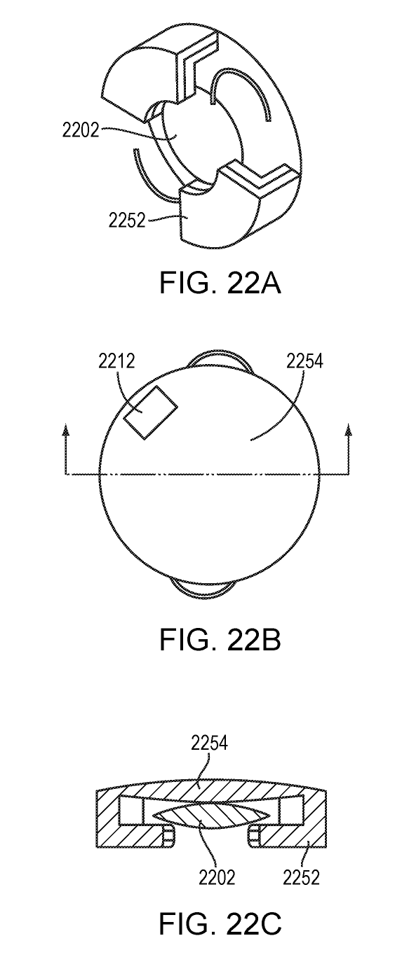

FIGS. 22A-22C depict the use of a “piggyback” lens component 2254. (FIG. 22C is a sectional view taken along the line A-A of FIG. 22B.) This piggyback lens component 2254 is most frequently used to correct refractive error or aberration from intraocular lens component 2202 after implantation. As an example, if intraocular lens component 2202 is implanted with an incorrect refractive power, the surgeon may place the piggyback lens to correct overall aberration. In certain circumstances this is less traumatic to the eye than intraocular lens exchange. In addition, piggyback lens component 2254 may have a valve 2212 to allow for adjusting the fit between the piggyback lens component 2254 and the IOL component 2202 as well as refraction of piggyback lens component 2254. Retention features 2252 may be used to directly couple and center piggyback lens component 2254 to IOL component 2202. Optionally, the retention features 2252 may be configured to retain the intraocular lens component 2202 through the fill adjustment process of the valve 2212.

The fluid-filled portions of the multiple-component implantable IOL are constructed of a biocompatible materials such as a polymer (e.g., parylene, silicone, silicone derivative such as a phenyl-substituted silicone, acrylic, polysulfone, hydrogel, collagen, or other suitable material). In certain embodiments, the membrane portions comprise or consist essentially of multiple materials (e.g., layered fluorosilicone and silicone, parylene deposited into or onto silicone, etc.). When a portion of the fluid-filled component acts as a lens, a biocompatible refractive filling fluid may be used. Examples of these fluids include but are not limited to oils such as silicone oil, fluorosilicone, phenyl-substituted silicone oil, perfluorocarbon, an aqueous material such as a sugar water, vegetable oil, gel, hydrogel, nanocomposite, or electrically active fluid. Other fluids include saline, ringers solution, or other aqueous solutions. In certain embodiments the chambers are filled with an osmotically active solute. By placing the component into the eye, the chamber fills through diffusion of aqueous fluid into the chamber. In other embodiments, the walls of the fluid-filled chambers are semipermeable to air and gas, allowing trapped air bubbles or gas to diffuse out over a period of time.

Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

What is claimed is:

1. An intraocular lens comprising:
a membrane defining a central chamber for containing an optical fluid and, when filled, to provide vision correction when implanted in a patient's eye, the central chamber having an optical axis extending through a vision-correcting optical zone of the central chamber; and
a spanning member extending between opposed areas of an internal surface of the membrane for resisting at least one of expansion or collapse of the central chamber.

2. The intraocular lens of claim 1, wherein the spanning member is elastomeric so as to restrain expansion of the membrane but not collapse thereof.

3. The intraocular lens of claim 1, wherein the spanning member is stiff so as to restrict both collapse and expansion of the membrane.

4. The intraocular lens of claim 1, wherein the spanning member is a spiral spring.

5. The intraocular lens of claim 1, wherein the spanning member extends along an optical axis of the lens.

6. The intraocular lens of claim 1, wherein the spanning member is continuous and solid.

7. The intraocular lens of claim 1, wherein the spanning member is tubular.

8. The intraocular lens of claim 7, wherein the lens has an optical zone and the spanning member has a diameter larger than a diameter of the optical zone of the lens.

9. The intraocular lens of claim 7, wherein the membrane is filled with a first optical fluid and the spanning member is filled with a second optical fluid.

10. The intraocular lens of claim 9, wherein the first and second optical fluids are the same.

11. The intraocular lens of claim 9, wherein the first and second optical fluids are different.

12. The intraocular lens of claim 1, wherein the membrane is filled with an optical fluid and the spanning member is permeable to the optical fluid.

13. The intraocular lens of claim 1, wherein the spanning member joins the internal surface of the membrane at first and second opposed ends.

14. The intraocular lens of claim 13, wherein each of the ends has at least one shaped terminal head member with a distal region attached to or integral with the interior surface of the membrane.

15. The intraocular lens of claim 14, wherein at least one of the head members has a terminal surface area sufficiently small relative to a surface area of the interior surface of the membrane to permit the membrane to bulge upon overfilling with an optical fluid.

16. The intraocular lens of claim 14, wherein at least one of the head members has a terminal surface area sufficiently large relative to a surface area of the interior surface of the membrane to resist bulging of the membrane upon overfilling with an optical fluid.

17. The intraocular lens of claim 14, wherein at least one end of the spanning member includes a plurality of branches each terminating in a head member with a distal region attached to or integral with the interior surface of the membrane.

18. The intraocular lens of claim 14, wherein at least one of the head members has a substantially symmetric terminal surface.

19. The intraocular lens of claim 14, wherein the terminal surface is round.

20. The intraocular lens of claim 14, wherein at least one of the head members has a substantially asymmetric terminal surface.

21. The intraocular lens of claim 14, wherein the terminal surface comprises a plurality of radial projections.

22. The intraocular lens of claim 14, wherein the exterior surface of the membrane overlying at least one of the head members has a plurality of radial grooves.

23. The intraocular lens of claim 1, wherein the spanning member is colored.

24.-80. (canceled)
